(19) United States
(12) Patent Application Publication
(10) Pub. No.: US 2007/0037192 A1
(43) Pub. Date: Feb. 15, 2007

(54) Method of purifying recombinant human antithrombin to enhance the viral and prion safety profile

(75) Inventors: Carol A. Ziomek, Milford, MA (US); Christopher Hendry, Medway, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: GTC Biotherapeutics, Inc., Framingham, MA (US)

(21) Appl. No.: 11/493,124

(22) Filed: Jul. 25, 2006

Related U.S. Application Data
(60) Provisional application No. 60/702,194, filed on Jul. 25, 2005.

(51) Int. Cl. C12Q 1/68; C12P 21/06

(52) U.S. Cl. 435/6; 435/69.1

(57) ABSTRACT

Methods of purifying antithrombin from a variety of source materials including from the milk of transgenic mammals to enhance its safety profile vis-à-vis the removal and/or the inactivation of contaminants. Contaminants would include particulate matter, viruses, and/or prions.

### FIELD OF THE INVENTION

The present invention relates to the transgenic production of recombinant human proteins which are biologically active and can be used to treat hereditary and acquired AT deficiencies and associated pathologies. In particular the current invention provides for the production of human antithrombin in the milk of transgenic mammals, particularly non-human placental mammals and provides for the use of such transgenic proteins in therapeutic applications or disease conditions.

### BACKGROUND OF THE INVENTION

As stated above, the present invention relates generally to the field of the transgenic production of transgenic proteins in the milk of transgenic animals. More particularly, it concerns improved methods for generating transgenic proteins capable of therapeutically treating hereditary and acquired deficiencies and related pathological conditions.

Antithrombin (“AT III or AT”) is a serine protease inhibitor, which inhibits thrombin and the activated forms of factors X, VII, IX, XI, and XII. It is normally present in serum at levels of 14-20 mg/dL. Current methods of obtaining plasma-derived AT involve isolating the protease inhibitor from blood plasma. However, the use of plasma-based AT presents various problems due to the many components in plasma, variation between lots and biohazardous risks due to viral and/or prion contamination. Therefore, a need exists to develop a method to produce recombinant antithrombin from a source without the inherent problems and risks of the present method in a process that is both more reliable and cost-efficient.

Accordingly, the recombinant processes of the current invention provide for the selective, as well as, more efficient methods of production of antithrombin that are needed to treat the incidence of hereditary and acquired AT deficiencies associated with the coagulation cascade and its associated pathologies. This may be accomplished, according to the current invention, through the use of rhAT in therapeutically effective amounts to reduce the incidence and severity of AT deficiency disorders.

### SUMMARY OF THE INVENTION

Briefly stated, the present invention relates generally to the production and purification of rhAT in a state that makes it available for therapeutic use.

This invention is also directed to compositions of purity and form that are suitable for pharmaceutical use and treatment of medical conditions. The purified form of rhAT, according to the invention will be comprised of the transgenic protein of interest, a prodrug thereof, or a pharmaceutically acceptable salt of said compound or of said prodrug and a pharmaceutically acceptable vehicle, diluent or carrier.

These and other objects which will be more readily apparent upon reading the following disclosure may be achieved by the present invention. Uses for recombinant rhAT include the use for treatment of hereditary and acquired AT deficiencies, including prophylaxis for prevention of DIC in hereditary deficient (HD) patients in high risk situations such as surgery or delivery. Additionally, rhAT may prove beneficial for various acquired AT deficiency states including but not limited to: DIC, burns, heparin resistance, neurocognitive deficit due to CABG surgery and sepsis.

In one aspect the invention provides a method for purifying a molecular species of interest from a feedstream, comprising: Filtering said feedstream by a tangential-flow filtration process (TFF) to produce a TFF permeate, cycling said TFF permeate through a closed loop system until at least 50% of the said molecular species of interest is captured wherein said closed loop system further comprises a Heparin-affinity column, nanofiltering said TFF/heparin eluate viral removal such that potential viral adventitious agents are removed, removing unwanted molecular contaminants through the use of an anion exchange column, utilizing a hydrophobic interaction column to eliminate unwanted or variant forms of said molecular species of interest, formulating said molecular species, lyophilizing said molecular species and heating the purified lyophilized molecular species of interest to inactive viruses. The method may further comprise lyophilizing said molecular species of interest after eluting this product from said hydrophobic interaction column. The method may further comprise a virus inactivation step providing dry heat of 80° C. for at least 72 hours. In one embodiment the heparin affinity column used in the method is a Heparin-Hyper D column. In other embodiments the TFF permeate is cycled through a closed loop system until at least 60% or 90% of said molecular species of interest is captured from a feedstream. In other embodiments the TFF permeate is cycled through a closed loop system for at least 5 or 8 volume cycles. In another embodiment the TFF permeate is cycled through said heparin affinity column, the retentate molecular species of interest is washed and then eluted with a first buffer. This first buffer may be a salt buffer further comprising a sodium chloride buffer, which may be 2.5 M sodium chloride. In another embodiment the anion column of the method is a sepharose column. In some embodiments the sepharose column is a ANX-Sepharose column. In some embodiments of the method after the use of the ANX-Sepharose column the molecular species of interest is eluted from the ANX-Sepharose column with a second buffer. This second buffer can be 0.32 M sodium chloride. In yet another embodiment of the method after the anion exchange column product eluate is collected and conditioned with sodium citrate. In some embodiments the conditioned product eluate is applied to a Methyl HyperD column and eluted with a third buffer. The third buffer can be a sodium citrate buffer. In some embodiments of the method the final formulation of the conditioned product eluate is achieved by concentration and diafiltration into a citrate, glycine, sodium chloride buffer. In some embodiments of the method the final protein concentration of the composition ranges from 20 IU/ml to 200 IU/ml. In some embodiments of the method the heat treatment further comprises heat treating lyophilized molecular species of interest at 80° C. for 72 hours in a viral inactivation step. In some embodiments of the method during said tangential flow filtration process the flux is maintained at a level ranging from about 5 to 100% of transition point flux in the pressure-dependent region of the flux versus TMP curve. In some embodiments of the method during the tangential flow filtration process the transmembrane pressure is held substantially constant along the membrane at a level no greater than the transmembrane pressure at the transition point of the filtration, whereby the molecular species of interest is selectively separated from the feedstream such that said molecular species of interest retains its biological activity. In some embodiments of the method the tangential-flow filtration process is performed through a filtration membrane having a pore size that separates said molecular species of interest from said feedstream. The filtration membrane may have a pore size of between 200 and 700 kD. In some embodiments the filtration membrane has a pore size of 500 kD. In some embodiments the filtration membrane is a 500 kD hollow fiber membrane. In yet another embodiment of the method prior to entering the purification process the feedstream can be diluted with an equal volume of an EDTA buffer. In some embodiments of the method the molecular species of interest is an antithrombin protein. In some embodiments of the method the purity of the molecular species of interest is at least 90%. In some embodiments of the method the purity of the molecular species of interest is greater than 99%. In some embodiments of the method the physiological activity of the molecular species of interest was at least 90%. In some embodiments of the method the physiological activity of the molecular species of interest was greater than 99%. In some embodiments of the method the purity of the molecular species is determined by SDS-PAGE or reverse phase-HPLC. In some embodiments of the method the molecular species of interest is a recombinant antithrombin protein. In some embodiments of the method the recombinant antithrombin is produced transgenically. In some embodiments of the method all filtration stages are ultrafiltrations. In some embodiments of the method the feedstream is milk. In some embodiments of the method the feedstream is a cell lysate solution. In some embodiments of the method the molecular species of interest is a biopharmaceutical. In some embodiments of the method the condition of the milk is selected from one of the following states: raw, diluted, treated with a buffer solution, chemically treated or partially evaporated. In some embodiments of the method the fractionation step and/or clarification step utilizes ceramic and/or polymeric and/or cellulose filtration membranes. In some embodiments the method further comprises optimizing systematic parameters. These parameters can include temperature, feedstream flow velocity, transmembrane pressure, feedstream concentration and diafiltration volume. In some embodiments of the method the systematic parameters may be optimized for the production of recombinant human antithrombin. In some embodiments of the method molecular species of interest are biological entities selected from the group consisting of proteins, polypeptides, peptides and glycoproteins, In some embodiments of the method the optimal temperature range is from 15° C. to 50° C., from 20° C. to 35° C., or from 25° C. to 29° C. In some embodiments of the method the feedstream flow velocity is from 10 cm/sec to 100 cm/sec, or from 20 cm/sec to 60 cm/sec, or from 25 cm/sec to 45 cm/sec. In some embodiments of the method the transmembrane pressure ranges from 2 psi to 40 psi, or from 5 psi to 30 psi, or from 10 psi to 20 psi. In some embodiments of the method the feedstream concentration is from 0.25× to 4× natural milk, or from 0.5× to 3× natural milk, or from 1.0× to 2× natural milk. In some embodiments of the method the diafiltration volume range is from 1× to 20× the volume of concentrated retentate, or from 3× to 15× the volume of concentrated retentate, or from 5× to 10× the volume of concentrated retentate. In some embodiments of the method the milk is treated with a solution selected from the group consisting of: water, a buffered aqueous salt solution, a chelating agent, an acid solution, or an alkali solution. In some embodiments the method further comprises filtering the filtrate from the filtration in a second tangential-flow filtration stage through a membrane having a smaller pore size than the membrane used in the first filtration stage, and recycling the filtrate of this second filtration stages back to the first filtration stage, whereby the process is repeated. In some embodiments of the method the pharmaceutical composition comprises reconstitution media selected from any one of polysorbate 20, polysorbate 21, polysorbate 40, polysorbate 60, polysorbate 61, polysorbate 65, polysorbate 80, polysorbate 81, polysorbate 85, polysorbate 120; and, human albumin. In some embodiments the reconstitution media is polysorbate 80, or human albumin. In one aspect the invention provides a method of purifying a recombinant antithrombin III (rhAT) or a fragment thereof from a feedstream, comprising solubilizing said rhAT from a feedstream utilizing a tangential flow filtration process, washing said filtrate on a membrane with a PBS solution wherein said rhAT or fragment thereof remains in the retentate on the purification column, adding an aqueous solution to said rhAT remaining in the retentate to solubilize it, eluting said rhAT from said purification column and purifying out said rhAT from elution. In another aspect the invention provides a method for purifying a molecular species of interest from a feedstream, comprising filtering said feedstream by a tangential-flow filtration process (TFF) to produce a TFF permeate, cycling said TFF permeate to through a closed loop system until at least 50% of the said molecular species of interest is captured wherein said closed loop system further comprises a Heparin-affinity column, collecting a first eluate from said Heparin-affinity column and processing said first eluate through a first concentration step and a first diafiltration step, transferring said first eluate into a downstream processing and formulation area thereafter again transferring said first eluate after said first concentration step and said first diafiltration step to a ion exchange chromatography column to generate a second eluate, removing unwanted molecular contaminants through the use of an anion exchange column, processing said second eluate through an anionic exchange chromatography column, nanofiltering said TFF permeate ion step for viral removal such that potential adventitious agents are removed, processing said second eluate through a second concentration step and a second diafiltration step to generate a third eluate, transferring said product eluate through a nanofilter capable of removing viruses from said third eluate, utilizing a hydrophobic interaction column to eliminate unwanted or variant forms of said molecular species of interest, processing said product eluate through a third concentration step and a third diafiltration step to generate a product eluate, loading said product eluate on a anion column and thereafter eluting with a buffer; and heating the purified molecular species of interest to inactive viruses. In some embodiments of the method the molecular species of interest is rhAT. In some embodiments the molecular species of interest is produced by any of the above methods In some embodiments the molecular species of interest is used therapeutically. In some embodiments the therapeutic condition treated is selected from the group consisting of: a hereditary rhAT deficiency, DIC, burns, heparin resistance, neurocognitive deficit due to CABG surgery and sepsis. In some embodiments of the method the resultant purified molecular species of interest is more than 90% from prion contamination found in normal milk, or transgenic milk. In some embodiments the resultant purified molecular species of interest is more than 90% from viral contamination found in normal milk or transgenic milk.

### BRIEF DESCRIPTION OF THE DRAWINGS

FIG. 1 Shows a Flowchart of an Embodiment of the Process of Creating Cloned Animals through Nuclear Transfer.

FIG. 2 Shows an Amino Acid Representation of Antithrombin.

FIG. 3 Shows the Construction of a cDNA Vector for Antithrombin.

FIG. 4 Shows a Method of Making a Transgenic Mammal.

FIG. 5 Shows a Process for the Purification of Recombinant Antithrombin from a Milk Feedstream.

FIG. 6 Shows the Purity Profile for the AT Process & Final Product According to the Methods of the Current Invention.

FIG. 7 Shows a Chromatogram of the Methyl Hyper D Elution Profiles of Goat and Human Antithrombin.

FIG. 7 Shows a Chromatogram of Methionine Oxidation in Thrombate, Kybernin and Recombinant Antithrombin.

FIG. 9 Shows a Non-Reducing Peptide Map of Thrombate and rhAT.

FIG. 10a & 10b Show Heparin Affinity for rhAT Before and After Glycosidase Treatment.

FIG. 11 Shows an Overview of rhAT Immunogenicity Analysis.

FIG. 12 Provides an Overview of Antithrombin's Role in the Coagulation Cascade.

FIG. 13 Provides an Overview of the Anti-Inflammatory Properties of AT.

FIG. 14 Shows the Pharmacodynamics of rhAT in Rats with Klebsiella Pneumoniae Induced Sepsis.

FIG. 15 Shows a filtration process flow diagram.

FIG. 16A Shows the process and equipment set-up for microfiltration.

FIG. 16B Shows the process and equipment set-up for TFF.

FIG. 17 Shows a process equipment schematic for the methods of the current invention.

FIG. 18 Shows the TFF process, mass balance as well as overall yield of the process according to the invention.

FIG. 19 Shows the ATryn® Manufacturing Process Flow Diagram.

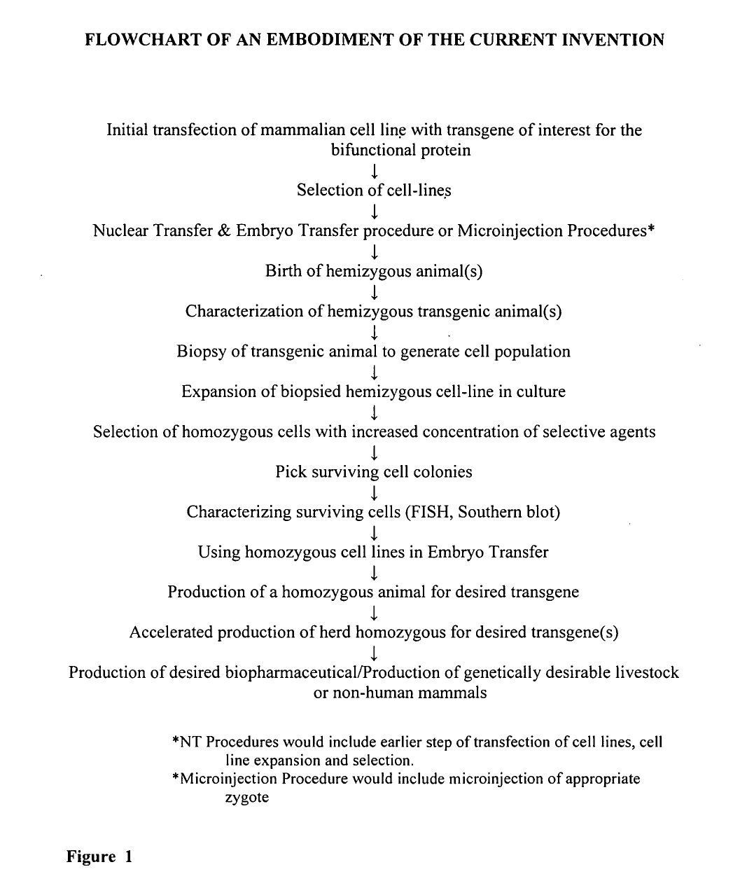

### DETAILED DESCRIPTION

The following abbreviations have designated meanings in the specification:

Abbreviation Key:
CV Crossflow Velocity
DFF Direct Flow Filtration
DV Diafiltration Volume
IEF Isoelectric Focusing
GMH Mass Flux (grams/m²/hour)—also J_{M}
LMH Liquid Flux (liters/m²/hour)—also J_{L}
LPM Liters Per Minute
M Molar
MF Microfiltration
NMWCO Nominal Molecular Weight Cut Off
NWP Normalized Water Permeability
PES Poly(ether)-sulfone
pH A term used to describe the hydrogen-ion activity of a chemical or compound according to well-known scientific parameters.
PPM Parts Per Million
SDS-PAGE SDS (sodium dodecyl sulfate) Poly-Acrylamide Gel electrophoresis
SEC Size Exclusion Chromatography
TFF Tangential Flow Filtration
PEG Polyethylene glycol
TMP Transmembrane Pressure
UF Ultrafiltration

Explanation of Terms:
Bovine—Of or relating to various species of cows.
Biological Fluid—an aqueous solution produced by an organism, such as a mammal, bird, amphibian, or reptile, which contains proteins that are secreted by cells that are bathed in the aqueous solution. Examples include: milk, urine, saliva, seminal fluid, vaginal fluid, synovial fluid, lymph fluid, amniotic fluid, blood, sweat, and tears; as well as an aqueous solution produced by a plant, including, for example, exudates and guttation fluid, xylem, phloem, resin, and nectar.
Biological-fluid producing cell—A cell that is bathed by a biological fluid and that secretes a protein into the biological fluid.
Biopharmaceutical—shall mean any medicinal drug, therapeutic, vaccine or any medically useful composition whose origin, synthesis, or manufacture involves the use of microorganisms, recombinant animals (including, without limitation, chimeric or transgenic animals), nuclear transfer, microinjection, or cell culture techniques.
Caprine—Of or relating to various species of goats.
Clarification—The removal of particulate matter from a solution so that the solution is able to pass through a 0.2 μm membrane.
Colloids—Refers to large molecules that do not pass readily across capillary walls. These compounds exert an oncotic (i.e., they attract fluid) load and are usually administered to restore intravascular volume and improve tissue perfusion.
Concentration—The removal of water and small molecules with a membrane such that the ratio of retained molecules to small molecules increases.
Concentration Polarization—The accumulation of the retained molecules (gel layer) on the surface of the membrane caused by a combination of factors: transmembrane pressure, crossflow velocity, sample viscosity, and solute concentration.
Crossflow Velocity—Velocity of the fluid across the top of the membrane surface. CF=Pi−Po where Pi is pressure at the inlet and Po is pressure at the outlet and is related to the retentate flow rate.
Diafiltration—The fractionation process of washing smaller molecules through a membrane, leaving the larger molecule of interest in the retentate. It is a convenient and efficient technique for removing or exchanging salts, removing detergents, separating free from bound molecules, removing low molecular weight materials, or rapidly changing the ionic or pH environment. The process typically employs a microfiltration membrane that is employed to remove a product of interest from a slurry while maintaining the slurry concentration as a constant.
Encoding—refers generally to the sequence information being present in a translatable form, usually operably linked to a promoter (e.g., a beta-casein or beta-lacto globulin promoter). A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code.
Expression Vector—A genetically engineered plasmid or virus, derived from, for example, a bacteriophage, adenovirus, retrovirus, poxvirus, herpesvirus, or artificial chromosome, that is used to transfer transgenic protein coding sequence, operably linked to a promoter, into a host cell, such that the encoded recombinant transgenic protein is expressed within the host cell.
Feedstream—The raw material or raw solution provided for a process or method and containing a protein of interest and which may also contain various contaminants including microorganisms, viruses and cell fragments.
Filtrate Flux (J)—The rate at which a portion of the sample has passed through the membrane.
Flow Velocity (V)—The speed at which the fluid passes the surface of the membrane is considered the fluid flow velocity. Product flux will be measured as flow velocity is varied. The relationship between the two variables will allow us to determine an optimal operational window for the flow.
Fractionation—The preferential separation of molecules based on a physical or chemical moiety.
Functional Proteins—Proteins which have a biological or other activity or use, similar to that seen when produced endogenously.
Gel Layer—The microscopically thin layer of molecules that can form on the top of a membrane. It can affect retention of molecules by clogging the membrane surface and thereby reduce the filtrate flow.
Milk-producing cell—A cell (e.g., a mammary epithelial cell) that secretes a protein into milk.
Molecule of Interest—Particles or other species of molecule that are to be separated from a solution or suspension in a fluid, e.g., a liquid. The particles or molecules of interest are separated from the fluid and, in most instances, from other particles or molecules in the fluid. The size of the molecule of interest to be separated will determine the pore size of the membrane to be utilized. Preferably, the molecules of interest are of biological or biochemical origin or produced by transgenic or in vitro processes and include proteins, peptides, polypeptides, antibodies or antibody fragments. Examples of preferred feedstream origins include mammalian milk, mammalian cell culture and microorganism cell culture such as bacteria, fungi, and yeast. It should also be noted that species to be filtered out include non-desirable polypeptides, proteins, cellular components, DNA, colloids, mycoplasm, endotoxins, viruses, carbohydrates, and other molecules of biological interest, whether glycosylated or not.
Nuclear Transfer—This refers to a method of cloning wherein the nucleus from a donor cell is transplanted into an enucleated oocyte.
Operably Linked—A gene and one or more regulatory sequences are connected in such a way as to permit gene expression when the appropriate molecules (e.g., transcriptional activator proteins) are bound to the regulatory sequences.
Ovine—Of or relating to or resembling sheep.
Pharmaceutically Pure—This refers to transgenic protein that is suitable for unequivocal biological testing as well as for appropriate administration to effect treatment of a human patient. Substantially pharmaceutically pure means at least about 90% pure.
Porcine—of or resembling pigs or swine.
Promoter—A minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.
Recombinant—refers to a nucleic acid sequence which is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functional polypeptide sequences to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., a human AT transgenic protein according to the instant invention.
Recovery—The amount of a molecule of interest that can be retrieved after processing. Usually expressed as a percentage of starting material or yield.
Retentate—The portion of the sample that does not pass through the membrane, also known as the concentrate. Retentate is being re-circulated during the TFF.
Tangential Flow Filtration—A process in which the fluid mixture containing the components to be separated by filtration is re-circulated at high velocities tangential to the plane of the membrane to increase the mass-transfer coefficient for back diffusion. In such filtrations a pressure differential is applied along the length of the membrane to cause the fluid and filterable solutes to flow through the filter. This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.
Therapeutically-effective amount—An amount of a therapeutic molecule or a fragment thereof that, when administered to a patient, inhibits or stimulates a biological activity modulated by that molecule.
Transformed cell or Transfected cell—A cell (or a descendent of a cell) into which a nucleic acid molecule encoding rhAT that has been introduced by means of recombinant DNA techniques. The nucleic acid molecule may be stably incorporated into the host chromosome, or may be maintained episomally.
Transgene—Any piece of a nucleic acid molecule that is inserted by artifice into a cell, or an ancestor thereof, and becomes part of the genome of the animal which develops from that cell. Such a transgene may include a gene which is partly or entirely exogenous (i.e., foreign) to the transgenic animal, or may represent a gene having identity to an endogenous gene of the animal.
Transgenic—Any cell that includes a nucleic acid molecule that has been inserted by artifice into a cell, or an ancestor thereof, and becomes part of the genome of the animal which develops from that cell.
Transgenic Organism—An organism into which genetic material from another organism has been experimentally transferred, so that the host acquires the genetic information of the transferred genes in its chromosomes in addition to that already in its genetic complement.
Transmembrane Pressure—The pressure differential gradient that is applied along the length of a filtration membrane to cause fluid and filterable solutes to flow through the filter. In tangential flow systems, highest TMP's are at the inlet (beginning of flow channel) and lowest at the outlet (end of the flow channel). TMP is calculated as an average pressure of the inlet, outlet, and filtrate ports.

According to the present invention, there is provided a method for the production of a transgenic protein of interest, the process comprising expressing in the milk of a transgenic non-human placental mammal a transgenic protein useful in the treatment of hereditary and acquired AT deficiencies or related pathologies and then processing the milk to remove the molecule of interest. The term “treating”, “treat” or “treatment” as used herein includes preventative (e.g., prophylactic) and palliative treatment.

Recombinant Production

To recombinantly produce a protein of interest a nucleic acid encoding a transgenic protein can be introduced into a host cell, e.g., a cell of a primary or immortalized cell line. The recombinant cells can be used to produce the transgenic protein, including a cell surface receptor that can be secreted from a mammary epithelial cell. A nucleic acid encoding a transgenic protein can be introduced into a host cell, e.g., by homologous recombination. In most cases, a nucleic acid encoding the transgenic protein of interest is incorporated into a recombinant expression vector.

The nucleotide sequence encoding a transgenic protein can be operatively linked to one or more regulatory sequences, selected on the basis of the host cells to be used for expression. The term “operably linked” means that the sequences encoding the transgenic protein compound are linked to the regulatory sequence(s) in a manner that allows for expression of the transgenic protein. The term “regulatory sequence” refers to promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990), the contents of which are incorporated herein by reference.

Regulatory sequences include those that direct constitutive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of transgenic protein desired, and the like. The transgenic protein expression vectors can be introduced into host cells to thereby produce transgenic proteins encoded by nucleic acids.

Recombinant expression vectors can be designed for expression of transgenic proteins in prokaryotic or eukaryotic cells. For example, transgenic proteins can be expressed in bacterial cells such as E. coli, insect cells (e.g., in the baculovirus expression system), yeast cells or mammalian cells. Some suitable host cells are discussed further in Goeddel, GENE EXPRESSION TECHNOLOGY: METHODS IN ENZYMOLOGY 185, Academic Press, San Diego, Calif. (1990). Examples of vectors for expression in yeast S. cerevisiae include pYepSecl (Baldari et al., (1987) EMBO J. 6:229-234), pMFa (Kurjan and Herskowitz, (1982) Cell 3:933-943), pJRY88 (Schultz et al., (1987) Gene 54:113-123), and pYES2 (Invitrogen Corporation, San Diego, Calif.). Baculovirus vectors available for expression of transgenic proteins in cultured insect cells include: the pAc series (Smith et al., (1983) MOL. CELL. BIOL. 3:2156-2165) and the pVL series (Lucklow, V. A., and Summers, M. D., (1989) VIROLOGY 170:31-39).

Examples of mammalian expression vectors include pCDM8 (Seed et al., (1987) NATURE 3:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and SV40.

In addition to the regulatory control sequences discussed above, the recombinant expression vector can contain additional nucleotide sequences. For example, the recombinant expression vector may encode a selectable marker gene to identify host cells that have incorporated the vector. Moreover, to facilitate secretion of the transgenic protein from a host cell, in particular mammalian host cells, the recombinant expression vector can encode a signal sequence operatively linked to sequences encoding the amino-terminus of the transgenic protein such that upon expression, the transgenic protein is synthesized with the signal sequence fused to its amino terminus. This signal sequence directs the transgenic protein into the secretory pathway of the cell and is then cleaved, allowing for release of the mature transgenic protein (i.e., the transgenic protein without the signal sequence) from the host cell. Use of a signal sequence to facilitate secretion of proteins or peptides from mammalian host cells is known in the art. Vector DNA can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

Transgenic Production of rhAT

Expression Construct and Production of Transgenic Founder Goat

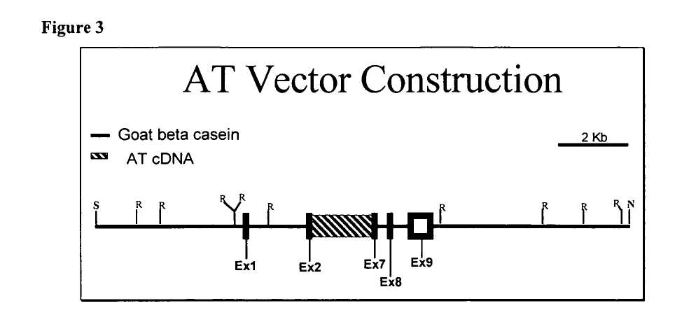

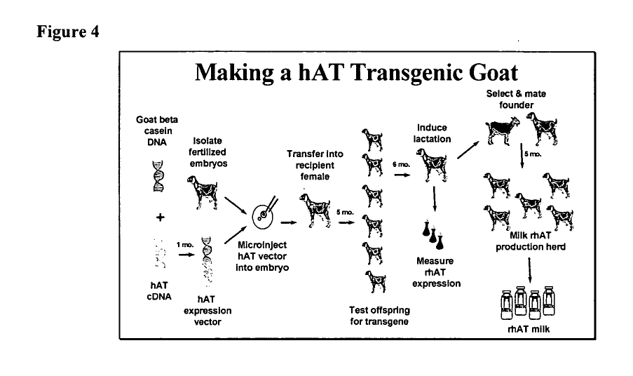

The hAT transgenic goats contain the cDNA for human AT (derived from a modified pBAT6 plasmid-Broker et al 1987) isolated from a human cDNA library in a goat beta casein expression cassette (FIG. 3). This milk specific expression cassette is routinely used by GTC for production of transgenic animals that express human therapeutic proteins in their milk. This expression construct was microinjected into pronuclei of fertilized goat eggs (FIG. 4), which were then transferred to the oviducts of pseudopregnant recipients. The gestation period in the goat is 150 days. The kids born were analyzed for the presence of the transgene and founder male 155-92 was so identified. Several generations of his female offspring have produced the milk used for purification of rhAT.

Transgenic Goats & Cattle

The herds of pure- and mixed-breed certified scrapie-free Alpine, Saanen and Toggenburg dairy goats used as embryo, semen, cell and cell line donors for this study were maintained under Good Agricultural Practices. Similarly, cattle used should be maintained under Good Agricultural Practices and be certified to originate from a BSE-free herd. Transgenic hAT founder goats were produced by microinjection of the transgene into the pronucleus of a fertilized caprine egg but could also have been produced by nuclear transfer technology.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/mL)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Transfected fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/mL) for at least 6 hours. The current experiments for the generation of desirable transgenic animals are preferably carried out with goat cells or mouse cells for the generation or goats or mice respectively but, according to the current invention, could be carried out with any mammalian cell line desired.

Oocyte Collection.

Oocyte donor does were synchronized and super ovulated as previously described (Ongeri, et al., 2001), and were mated over a 48-hour interval to fertile males for microinjection procedures and to vasectomized males for nuclear transfer procedures. After collection, fertilized embryos or unfertilized oocytes were cultured in equilibrated M199 with 10% FBS supplemented with 2 mM L-glutamine and 1% penicillin/streptomycin (10,000 I.U. each/mL).

Cytoplast Preparation and Enucleation.

All oocytes were treated with cytochalasin-B (Sigma, 5 μg/mL in SOF with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in SOF for 30 to 60 minutes before electrotransgenic and activation procedures. Reconstructed oocytes were equilibrated in transgenic buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electro-fusion and activation were conducted at room temperature, in a transgenic chamber with 2 stainless steel electrodes fashioned into a “transgenic slide” (500 μm gap; BTX-Genetronics, San Diego, Calif.) filled with transgenic medium.

Transgenic fusion was performed using a transgenic slide. The transgenic slide was placed inside a transgenic dish, and the dish was flooded with a sufficient amount of transgenic buffer to cover the electrodes of the transgenic slide. Couplets were removed from the culture incubator and washed through transgenic buffer. Using a stereomicroscope, couplets were placed equidistant between the electrodes, with the karyoplast/cytoplast junction parallel to the electrodes. It should be noted that the voltage range applied to the couplets to promote activation and transgenic fusion can be from 1.0 kV/cm to 10.0 kV/cm. Preferably however, the initial single simultaneous transgenic and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh transgenic buffer. Transgenic treated couplets were washed through equilibrated SOF/FBS, then transferred to equilibrated SOF/FBS with or without cytochalasin-B. If cytocholasin-B is used its concentration can vary from 1 to 15 μg/mL, most preferably at 5 μg/mL. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Meng et al., 1997), have also been reported. The stage of cell cycle that the karyoplast is in at time of reconstruction has also been documented as critical in different laboratories methodologies (Kasinathan et al., BIOL. REPROD. 2001; Yong et al., 1998; and Kasinathan et al., NATURE BIOTECH. 2001).

All nuclear transfer embryos of the current invention were cultured in 50 μL droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Similarly, known microinjection protocols can be utilized to produce a transgenic animal contemplated by the invention and capable of producing rhAT.

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2μ (Lutalyse, Upjohn). Parturition occurred within 24 hours after treatment. Kids were removed from the dam immediately after birth, and received heat-treated colostrum within 1 hour after delivery. Time frames appropriate for other ungulates with regard to pregnancy and perinatal care (e.g., bovines) are known in the art.

Cloned Animals.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte. Also provided by the present invention are mammals obtained according to the above method, and the offspring of those mammals. The present invention is preferably used for cloning caprines or bovines but could be used with any mammalian species. The present invention further provides for the use of nuclear transfer fetuses and nuclear transfer and chimeric offspring in the area of cell, tissue and organ transplantation.

Suitable mammalian sources for oocytes include goats, sheep, cows, pigs, rabbits, guinea pigs, mice, hamsters, rats, primates, etc. Preferably, the oocytes will be obtained from ungulates, and most preferably goats or cattle. Methods for isolation of oocytes are well known in the art. Essentially, this will comprise isolating oocytes from the ovaries or reproductive tract of a mammal, e.g., a goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, transgenic proteins, plasma proteins, and other molecules of interest in the milk or other bodily fluid (i.e., urine or blood) of transgenic animals hemizygous for a desired gene.

The use of living organisms as the production process means that all of the material produced will be identical in amino-acid sequence to the natural product. In terms of basic amino acid structures this means that only L-optical isomers, having the natural configuration, will be present in the product. Also the number of wrong sequences will be negligible because of the high fidelity of biological synthesis compared to chemical routes, in which the relative inefficiency of coupling reactions will always produce failed sequences. The absence of side reactions is also an important consideration with further modification reactions such as carboxy-terminal amidation. Again, the enzymes operating in vivo give a high degree of fidelity and stereospecificity which cannot be matched by chemical methods. Finally the production of a transgenic protein of interest in a biological fluid means that low-level contaminants remaining in the final product are likely to be far less toxic than those originating from a chemical reactor.

As previously mentioned, expression levels of several grams per liter of caprine milk are well within the reach of existing transgenic animal technology. Such levels should also be achievable for the recombinant protein contemplated by the current invention.

In the practice of the present invention, recombinant human antithrombin is produced in the milk of transgenic animals. The human recombinant protein of interest coding sequences can be obtained by screening libraries of genomic material or reverse-translated messenger RNA derived from the animal of choice (such as cattle or mice), or through appropriate sequence databases such as NCBI, genbank, etc. These sequences along with the desired polypeptide sequence of the transgenic partner protein are then cloned into an appropriate plasmid vector and amplified in a suitable host organism, usually E. coli. The DNA sequence encoding the peptide of choice can then be constructed, for example, by polymerase chain reaction amplification of a mixture of overlapping annealed oligonucleotides.

After amplification of the vector, the DNA construct would be excised with the appropriate 5′ and 3′ control sequences, purified away from the remains of the vector and used to produce transgenic animals that have integrated into their genome the desired transgenic protein. Conversely, with some vectors, such as yeast artificial chromosomes (YACs), it is not necessary to remove the assembled construct from the vector; in such cases the amplified vector may be used directly to make transgenic animals. In this case refers to the presence of a first polypeptide encoded by enough of a protein nucleic acid sequence to retain its biological activity, this first polypeptide is then joined to a the coding sequence for a second polypeptide also containing enough of a polypeptide sequence of a protein to retain its physiological activity. The coding sequence being operatively linked to a control sequence which enables the coding sequence to be expressed in the milk of a transgenic non-human placental mammal.

A DNA sequence which is suitable for directing production to the milk of transgenic animals carries a 5′-promoter region derived from a naturally-derived milk protein and is consequently under the control of hormonal and tissue-specific factors. Such a promoter should therefore be most active in lactating mammary tissue. According to the current invention the promoter so utilized can be followed by a DNA sequence directing the production of a protein leader sequence which would direct the secretion of the transgenic protein across the mammary epithelium into the milk. At the other end of the transgenic protein construct a suitable 3′-sequence, preferably also derived from a naturally secreted milk protein, may be added to improve stability of mRNA. An example of suitable control sequences for the production of proteins in the milk of transgenic animals are those from the caprine beta casein promoter.

The production of transgenic animals can now be performed using a variety of methods. The methods preferred by the current invention is microinjection or nuclear transfer.

Milk Specific Promoters.

The transcriptional promoters useful in practicing the present invention are those promoters that are preferentially activated in mammary epithelial cells, including promoters that control the genes encoding milk proteins such as caseins, beta-lacto globulin (Clark et al., (1989) BIO/TECHNOLOGY 7: 487-492), whey acid protein (Gorton et al. (1987) BIO/TECHNOLOGY 5: 1183-1187), and lactalbumin (Soulier et al., (1992) FEBS LETTS. 297: 13). Casein promoters may be derived from the alpha, beta, gamma or kappa casein genes of any mammalian species; a preferred promoter is derived from the goat beta casein gene (DiTullio, (1992) BIO/TECHNOLOGY 10:74-77). The milk-specific protein promoter or the promoters that are specifically activated in mammary tissue may be derived from either cDNA or genomic sequences. Preferably, they are genomic in origin.

DNA sequence information is available for all of the mammary gland specific genes listed above, in at least one, and often several organisms. See, e.g., Richards et al., J. BIOL. CHEM. 256, 526-532 (1981) (α-lactalbumin rat); Campbell et al., NUCLEIC ACIDS RES. 12, 8685-8697 (1984) (rat WAP); Jones et al., J. BIOL. CHEM. 260, 7042-7050 (1985) (rat β-casein); Yu-Lee & Rosen, J. BIOL. CHEM. 258, 10794-10804 (1983) (rat γ-casein); Hall, BIOCHEM. J. 242, 735-742 (1987) (α-lactalbumin human); Stewart, NUCLEIC ACIDS RES. 12, 389 (1984) (bovine αs1 and κ casein cDNAs); Gorodetsky et al., GENE 66, 87-96 (1988) (bovine β casein); Alexander et al., EUR. J. BIOCHEM. 178, 395-401 (1988) (bovine κ casein); Brignon et al., FEBS LETT. 188, 48-55 (1977) (bovine αS2 casein); Jamieson et al., GENE 61, 85-90 (1987), Ivanov et al., BIOL. CHEM. Hoppe-Seyler 369, 425-429 (1988), Alexander et al., NUCLEIC ACIDS RES. 17, 6739 (1989) (bovine β lactoglobulin); Vilotte et al., BIOCHIMIE 69, 609-620 (1987) (bovine α-lactalbumin). The structure and function of the various milk protein genes are reviewed by Mercier & Vilotte, J. DAIRY SCI. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

Signal Sequences.

Among the signal sequences that are useful in accordance with this invention are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin are useful in the present invention. The preferred signal sequence is the goat β-casein signal sequence.

Signal sequences from other secreted proteins, e.g., proteins secreted by liver cells, kidney cell, or pancreatic cells can also be used.

Amino-Terminal Regions of Secreted Proteins.

The efficacy with which a non-secreted protein is secreted can be enhanced by inclusion in the protein to be secreted all or part of the coding sequence of a protein which is normally secreted. Preferably the entire sequence of the protein which is normally secreted is not included in the sequence of the protein but rather only a portion of the amino terminal end of the protein which is normally secreted. For example, a protein which is not normally secreted is fused (usually at its amino terminal end) to an amino terminal portion of a protein which is normally secreted.

Preferably, the protein which is normally secreted is a protein which is normally secreted in milk. Such proteins include proteins secreted by mammary epithelial cells, milk proteins such as caseins, beta lacto globulin, whey acid protein, and lactalbumin. Casein proteins include alpha, beta, gamma or kappa casein genes of any mammalian species. A preferred protein is beta casein, e.g., a goat beta casein. The sequences which encode the secreted protein can be derived from either cDNA or genomic sequences. Preferably, they are genomic in origin, and include one or more introns.

DNA Constructs.

The expression system or construct, described herein, can also include a 3′ untranslated region downstream of the DNA sequence coding for the non-secreted protein. This region apparently stabilizes the RNA transcript of the expression system and thus increases the yield of desired protein from the expression system. Among the 3′ untranslated regions useful in the constructs of this invention are sequences that provide a poly A signal. Such sequences may be derived, e.g., from the SV40 small t antigen, the casein 3′ untranslated region or other 3′ untranslated sequences well known in the art. Preferably, the 3′ untranslated region is derived from a milk specific protein. The length of the 3′ untranslated region is not critical but the stabilizing effect of its poly A transcript appears important in stabilizing the RNA of the expression sequence.

Optionally, the expression system or construct includes a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of the gene preferentially expressed in mammary epithelial cells. For example, the N-terminal coding region can correspond to the promoter used, e.g., a goat β-casein N-terminal coding region.

The above-described expression systems may be prepared using methods well known in the art. For example, various ligation techniques employing conventional linkers, restriction sites etc. may be used to good effect. Preferably, the expression systems of this invention are prepared as part of larger plasmids. Such preparation allows the cloning and selection of the correct constructions in an efficient manner as is well known in the art. Most preferably, the expression systems of this invention are located between convenient restriction sites on the plasmid so that they can be easily isolated from the remaining plasmid sequences for incorporation into the desired mammal.

Prior art methods often include making a construct and testing it for the ability to produce a product in cultured cells prior to placing the construct in a transgenic animal. Surprisingly, the inventors have found that such a protocol may not be of predictive value in determining if a normally non-secreted protein can be secreted, e.g., in the milk of a transgenic animal. Therefore, it may be desirable to test constructs directly in transgenic animals, e.g., transgenic mice, as some constructs which fail to be secreted in CHO cells are secreted into the milk of transgenic animals.

Sequence Production and Modification

The invention encompasses the use of the described nucleic acid sequences and the peptides expressed therefrom in various transgenic animals. The sequences of specific molecules can be manipulated to generate proteins that retain most of their tertiary structure but are physiologically non-functional.

PCR technology may also be utilized to isolate full length cDNA sequences. For example, RNA may be isolated, following standard procedures, from an appropriate cellular or tissue source (i.e., one known, or suspected, to express a target receptor gene, such as, for example from skin, testis, or brain tissue). A reverse transcription (RT) reaction may be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment may easily be isolated. For a review of cloning strategies which may be used, see e.g., Sambrook et al., 1989.

A cDNA of a mutant target gene may be isolated, for example, by using PCR. In this case, the first cDNA strand may be synthesized by hybridizing an oligo-dT oligonucleotide to mRNA isolated from tissue known or suspected to be expressed in an individual putatively carrying a mutant target allele, and by extending the new strand with reverse transcriptase. The second strand of the cDNA is then synthesized using an oligonucleotide that hybridizes specifically to the 5′ end of the normal gene. Using these two primers, the product is then amplified via PCR, optionally cloned into a suitable vector, and subjected to DNA sequence analysis through methods well known to those of skill in the art. By comparing the DNA sequence of the mutant target allele to that of the normal target allele, the mutation(s) responsible for the loss or alteration of function of the mutant target gene product can be ascertained.

Alternatively, a genomic library can be constructed using DNA obtained from an individual suspected of or known to carry the mutant target allele, or a cDNA library can be constructed using RNA from a tissue known, or suspected, to express the mutant target allele. A normal target gene, or any suitable fragment thereof, can then be labeled and used as a probe to identify the corresponding mutant target allele in such libraries. Clones containing the mutant target gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant target allele in an individual suspected of or known to carry such a mutant allele. In this manner, gene products made by the putatively mutant tissue may be expressed and screened using standard antibody screening techniques in conjunction with antibodies raised against the normal target product.

The target protein amino acid sequences of the invention include the amino acid sequences presented in the sequence listings herein as well as analogues and derivatives thereof. Further, corresponding target protein homologues from other species are encompassed by the invention. The degenerate nature of the genetic code is well known, and, accordingly, each amino acid presented in the sequence listings, is generically representative of the well known nucleic acid “triplet” codon, or in many cases codons, that can encode the amino acid. As such, as contemplated herein, the amino acid sequences presented in the sequence listing, when taken together with the genetic code (see, pp 109, Table 4-1 of MOLECULAR CELL BIOLOGY, (1986), J. Darnell et al. eds., incorporated by reference) are generically representative of all the various permutations and combinations of nucleic acid sequences that can encode such amino acid sequences.

According to a preferred embodiment of the invention random mutations can be made to target gene DNA through the use of random mutagenesis techniques well known to those skilled in the art with the resulting mutant target proteins tested for activity, site-directed mutations of the target protein coding sequence can be engineered to generate mutant target receptor proteins with the same structure but with limited physiological function, e.g., alternate function, and/or with increased half-life. This can be accomplished using site-directed mutagenesis techniques well known to those skilled in the art.

One starting point for such activities is to align the disclosed human sequences with corresponding gene/protein sequences from, for example, other mammals in order to identify specific amino acid sequence motifs within the target gene that are conserved between different species. Changes to conserved sequences can be engineered to alter function, signal transduction capability, or both. Alternatively, where the alteration of function is desired, deletion or non-conservative alterations of the conserved regions can also be engineered.

Other mutations to the target protein coding sequence can be made to generate target proteins that are better suited for expression, scale-up, etc. in the host cells chosen. For example, cysteine residues can be deleted or substituted with another amino acid in order to eliminate disulfide bridges.

While the target proteins and peptides can be chemically synthesized, large sequences derived from a target protein and full length gene sequences can be advantageously produced by recombinant DNA technology using techniques well known in the art for expressing nucleic acid containing target protein gene sequences and/or nucleic acid coding sequences. Such methods can be used to construct expression vectors containing appropriate transcriptional and translational control signals. These methods include, for example, in vitro recombinant DNA techniques, synthetic techniques, and in vivo genetic recombination.

Transgenic Mammals.

Preferably, the DNA constructs of the invention are introduced into the germ-line of a mammal. For example, one or several copies of the construct may be incorporated into the genome of a mammalian embryo by standard transgenic techniques known in the art.

Any non-human mammal can be usefully employed in this invention. Mammals are defined herein as all animals, excluding humans, which have mammary glands and produce milk. Preferably, mammals that produce large volumes of milk and have long lactating periods are preferred. Preferred mammals are cows, sheep, goats, mice, oxen, camels and pigs. Of course, each of these mammals may not be as effective as the others with respect to any given expression sequence of this invention. For example, a particular milk-specific promoter or signal sequence may be more effective in one mammal than in others. However, one of skill in the art may easily make such choices by following the teachings of this invention.

In an exemplary embodiment of the current invention, a transgenic non-human animal is produced by introducing a transgene into the germline of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

The litters of transgenic mammals may be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity. The female species of these progeny will produce the desired protein in or along with their milk. Alternatively, the transgenic mammals may be bred to produce other transgenic progeny useful in producing the desired proteins in their milk.

In accordance with the methods of the current invention for transgenic animals a transgenic primary cell line (from either caprine, bovine, ovine, porcine or any other non-human vertebrate origin) suitable for somatic cell nuclear transfer is created by transfection of the transgenic protein nucleic acid construct of interest (for example, a mammary gland-specific transgene(s) targeting expression of a transgenic protein to the mammary gland). The transgene construct can either contain a selection marker (such as neomycin, kanamycin, tetracycline, puromycin, zeocin, hygromycin or any other selectable marker) or be co-transfected with a cassette able to express the selection marker in cell culture.

Transgenic females may be tested for protein secretion into milk, using any of the assay techniques that are standard in the art (e.g., Western blots or enzymatic assays).

The invention provides expression vectors containing a nucleic acid sequence described herein, operably linked to at least one regulatory sequence. Many such vectors are commercially available, and other suitable vectors can be readily prepared by the skilled artisan. “Operably linked” or “operatively linked” is intended to mean that the nucleic acid molecule is linked to a regulatory sequence in a manner which allows expression of the nucleic acid sequence by a host organism. Regulatory sequences are art recognized and are selected to produce the encoded polypeptide or protein. Accordingly, the term “regulatory sequence” includes promoters, enhancers, and other expression control elements which are described in Goeddel, GENE EXPRESSION TECHNOLOGY: METHODS IN ENZYMOLOGY 185, (Academic Press, San Diego, Calif. (1990)). For example, the native regulatory sequences or regulatory sequences native to the transformed host cell can be employed.

It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both. (A LABORATORY MANUAL, 2nd Ed., ed. Sambrook et al. (Cold Spring Harbor Laboratory Press, 1989) Chap. 16-17).

Following selection of colonies recombinant for the desired nucleic acid construct, cells are isolated and expanded, with aliquots frozen for long-term preservation according to procedures known in the field. The selected transgenic cell-lines can be characterized using standard molecular biology methods (PCR, Southern blotting, FISH). Cell lines carrying nucleic acid constructs of the obesity related transgenic protein of interest, of the appropriate copy number, generally with a single integration site (although the same technique could be used with multiple integration sites) can then be used as karyoplast donors in a somatic cell nuclear transfer protocol known in the art. Following nuclear transfer, and embryo transfer to a recipient animal, and gestation, live transgenic offspring are obtained.

Typically this transgenic offspring carries only one transgene integration on a specific chromosome, the other homologous chromosome not carrying an integration in the same site. Hence the transgenic offspring is hemizygous for the transgene, maintaining the current need for at least two successive breeding cycles to generate a homozygous transgenic animal.

Animal Promoters

Useful promoters for the expression of a target protein the mammary tissue include promoters that naturally drive the expression of mammary-specific polypeptides, such as milk proteins. These include, e.g., promoters that naturally direct expression of whey acidic protein (WAP), alpha S1-casein, alpha S2-casein, beta-casein, kappa-casein, beta-lactoglobulin, alpha-lactalbumin (see, e.g., Drohan et al., U.S. Pat. No. 5,589,604; Meade et al., U.S. Pat. No. 4,873,316; and Karatzas et al., U.S. Pat. No. 5,780,009), and others described in U.S. Pat. No. 5,750,172. Whey acidic protein (WAP; Genbank Accession No. X01153), the major whey protein in rodents, is expressed at high levels exclusively in the mammary gland during late pregnancy and lactation (Hobbs et al., J. BIOL. CHEM. 257:3598-3605, 1982). For additional information on desired mammary gland-specific promoters, see, e.g., Richards et al., J. BIOL. CHEM. 256:526-532, 1981 (α-lactalbumin rat); Campbell et al., NUCLEIC ACIDS RES. 12:8685-8697, 1984 (rat WAP); Jones et al., J. BIOL. CHEM. 260:7042-7050, 1985 (rat β-casein); Yu-Lee & Rosen, J. BIOL. CHEM. 258:10794-10804, 1983 (rat γ-casein); Hall, BIOCHEM. J. 242:735-742, 1987 (human α-lactalbumin); Stewart, NUCLEIC ACIDS RES. 12:3895-3907, 1984 (bovine α-sl and κ-casein cDNAs); Gorodetsky et al., GENE 66:87-96, 1988 (bovine β-casein); Alexander et al., EUR. J. BIOCHEM. 178:395-401, 1988 (bovine κ-casein); Brignon et al., FEBS LETT. 188:48-55, 1977 (bovine α-S2 casein); Jamieson et al., GENE 61:85-90, 1987, Ivanov et al., BIOL. CHEM. Hoppe-Seyler 369:425-429, 1988, and Alexander et al., NUCLEIC ACIDS RES. 17:6739, 1989 (bovine β-lactoglobulin); and Vilotte et al., BIOCHIMIE 69:609-620, 1987 (bovine α-lactalbumin). The structure and function of the various milk protein genes are reviewed by Mercier & Vilotte, J. DAIRY SCI. 76:3079-3098, 1993.

Other promoters that are useful in the methods of the invention include inducible promoters. Generally, recombinant proteins are expressed in a constitutive manner in most eukaryotic expression systems. The addition of inducible promoters or enhancer elements provides temporal or spatial control over expression of the transgenic proteins of interest, and provides an alternative mechanism of expression. Inducible promoters include heat shock protein, metallothionien, and MMTV-LTR, while inducible enhancer elements include those for ecdysone, muristerone A, and tetracycline/doxycycline.

Therapeutic Uses.

The combination herein is preferably employed for in vitro use in treating these tissue cultures. The combination, however, is also effective for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute lyophilized a human recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier will be amounts which are effective to obtain a pharmaceutically acceptable formulation in terms of solubility of components, biological activity, etc.

The compositions herein may be administered to human patients via oral, parenteral or topical administrations.

Bacterial Expression.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may, also be employed as a matter of choice. In a preferred embodiment, the prokaryotic host is E. coli.

Bacterial vectors may be, for example, bacteriophage-, plasmid- or cosmid-based. These vectors can comprise a selectable marker and bacterial origin of replication derived from commercially available plasmids typically containing elements of the well known cloning vector pBR322 (ATCC 37017). Such commercial vectors include, for example, GEM 1 (Promega Biotec, Madison, Wis., USA), pBs, phagescript, PsiX174, pBluescript SK, pBs KS, pNH8a, pNH16a, pNH18a, pNH46a (Stratagene); pTrc99A, pKK223-3, pKK233-3, pKK232-8, pDR540, and pRIT5 (Pharmacia).

These “backbone” sections are combined with an appropriate promoter and the structural sequence to be expressed. Bacterial promoters include lac, T3, T7, lambda PR or PL, trp, and ara. T7 is a preferred bacterial promoter.

Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

Eukaryotic Expression Vectors

Various mammalian cell culture systems can also be employed to express recombinant proteins. Examples of mammalian expression systems include selected mouse L cells, such as thymidine kinase-negative (TK) and adenine phosphoribosul transferase-negative (APRT) cells. Other examples include the COS-7 lines of monkey kidney fibroblasts, described by Gluzman, CELL 23:175 (1981), and other cell lines capable of expressing a compatible vector, for example, the C127, 3T3, CHO, HeLa and BHK cell lines. In particular, as regards yeasts, there may be mentioned yeasts of the genus Saccharomyces, Kluyveromyces, Pichia, Schwanniomyces, or Hansenula. Among the fungi capable of being used in the present invention, there may be mentioned more particularly Aspergillus ssp, or Trichoderma ssp.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements.

Mammalian promoters include beta-casein, beta-lactoglobulin, whey acid promoter others include: HSV thymidine kinase, early and late SV40, LTRs from retrovirus, and mouse metallothionein-1. Exemplary mammalian vectors include pWLneo, pSV2cat, pOG44, pXT1, pSG (Stratagene) pSVK3, pBPV, pMSG, and pSVL (Pharmacia). In a preferred embodiment, the mammalian expression vector is pUCIG-MET. Selectable markers include CAT (chloramphenicol transferase).

Therapeutic Compositions.

The proteins of the present invention can be formulated according to known methods to prepare pharmaceutically useful compositions, whereby the inventive molecules, or their functional derivatives, are combined in admixture with a pharmaceutically acceptable carrier vehicle. Suitable vehicles and their formulation, inclusive of other human proteins, e.g., human serum albumin, are described, for example, in order to form a pharmaceutically acceptable composition suitable for effective administration, such compositions will contain an effective amount of one or more of the proteins of the present invention, together with a suitable amount of carrier vehicle.

Pharmaceutical compositions for use in accordance with the present invention may be formulated in conventional manner using one or more physiologically acceptable carriers or excipients.

The transgenic proteins of the invention may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may, if desired, be presented in a pack or dispenser device which may contain one or more unit dosage forms containing the active ingredient. The pack or dispenser device may be accompanied by instructions for administration.

Treatment Methods.

Therapeutic methods involve administering to a subject in need of treatment a therapeutically effective amount of a transgenic protein. “Therapeutically effective” is employed here to denote the amount of transgenic proteins that are of sufficient quantity to inhibit or reverse a disease condition (e.g., reduce or inhibit hereditary or acquired deficiency). Administration during in vivo treatment is by the intravenous and/or intraperitoneal routes of administration.

Determining a therapeutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious.

The Purification Process

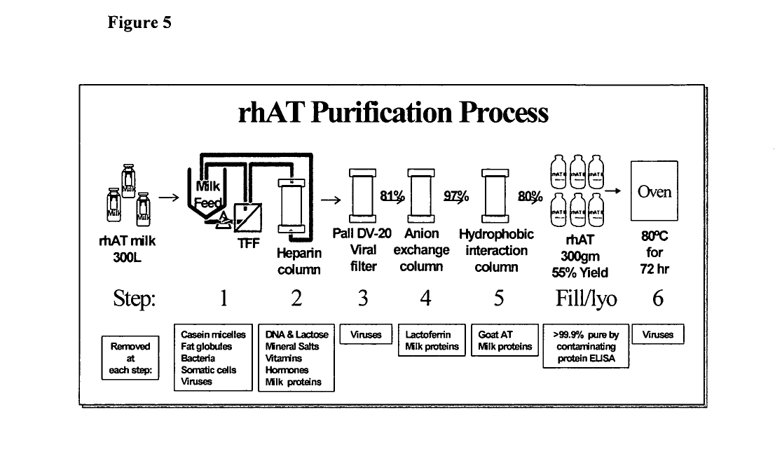

The source material for purification is milk produced by transgenic goats expressing the rhAT protein at approximately 2 g/l. Goats typically lactate 300 days per year producing greater than 1 liter of milk per day. The purification process normally produces 300 grams of purified rhAT per batch from no more than 375 liters of milk containing approximately 600 grams of rhAT. The volume of milk processed per batch is determined based on the rhAT binding capacity of the initial Heparin (Heparin-Hyper D) column and the rhAT concentration of the milk. The full rhAT purification process is depicted in FIG. 5.

Upstream processing is defined as thawing, pooling and clarification of the source material (milk) and initial mass capture of the substances that will become the feedstream. Milk containing rhAT is diluted with an equal weight of EDTA buffer and is then clarified by tangential flow filtration with a nominal 500-kDa-pore size Hollow Fiber membrane filter (Step 1). The 500-kDa filter permeate is cycled through a closed loop linking the filtration system to the Heparin-Hyper D column (Step 2) until >90% of the rhAT is captured (about 8 volume cycles). The Heparin-Hyper D column is washed and then eluted with a 2.5 M sodium chloride buffer. Once the Heparin-Hyper D eluate is obtained, it is transferred into a downstream processing and formulation area.

Downstream processing (FIG. 5) includes concentration and diafiltration of the Heparin-Hyper D column eluate, ion exchange chromatography (ANX-Sepharose), hydrophobic interaction chromatography (Methyl HyperD) and a second concentration and diafiltration step. After the heparin eluate is transferred into the downstream processing area, it is filtered through a Pall DV-20 viral removal filter, concentrated and diafiltered by membrane filtration to adjust the ionic strength for the application of the rhAT onto the ANX-Sepharose column. After loading, the ANX-Sepharose column is washed and the rhAT is eluted with 0.32 M buffer. The ANX-Sepharose eluate is conditioned with sodium citrate and applied to the Methyl HyperD column. The Methyl HyperD column is washed and the rhAT is eluted from the resin with a lower concentration sodium citrate buffer.

Final formulation is achieved by concentration and diafiltration into a citrate, glycine, sodium chloride buffer with the proper ionic strength and dilution to the final preferred protein concentration of approximately 25 mg/m or an activity of 175 IU/ml. Formulated batches are tested, and those meeting the product specifications may be pooled if desired to meet the lot size requirements. The product is filled into vials (10 ml containing approximately 250 mg protein), lyophilized and then heat treated in a validated controlled temperature oven, according to a preferred embodiment, at 80° C. for 72 hours in a validated terminal viral inactivation step.

Tangential Flow Filtration

There are two important variables involved in all tangential flow devices: the transmembrane pressure (TMP) and the crossflow velocity (CF). The transmembrane pressure (TMP) is the force that actually pushes molecules through the pores of the filter. The crossflow velocity is the flow rate of the solution across the membrane. It provides the force that sweeps away larger molecules that can clog the membrane thereby reducing the effectiveness of the process. In practice a fluid feedstream is pumped from the sample feed container source across the membrane surface (crossflow) in the filter and back into the sample feed container as the retentate. Backpressure applied to the retentate tube by a clamp creates a transmembrane pressure which drives molecules smaller than the membrane pores through the filter and into the filtrate (or permeate) fraction. The crossflow sweeps larger molecules, which are retained on the surface of the membrane, back to the feed as retentate. The primary objective for the successful implementation of a TFF protocol is to optimize the TMP and CF so that the largest volume of sample can be filtered without creating a membrane-clogging gel. A TMP is “substantially constant” if the TMP does not increase or decrease along the length of the membrane generally by more than about 10 psi of the average TMP, and preferably by more than about 5 psi. As to the level of the TMP throughout the filtration, the TMP is held constant or is lowered during the concentration step to retain selectivity at higher concentrations. Thus, “substantially constant TMP” refers to TMP versus membrane length, not versus filtration time.

Milk as a Feedstream

According to a preferred embodiment of the current invention, the TFF process employs three filtration unit operations that clarify, concentrate, and fractionate the product from a milk feedstream. This milk may be the product of a transgenic mammal containing a biopharmaceutical or other molecule of interest. In a preferred embodiment the system is designed such that it is highly selective for the molecule of interest. The clarification step removes larger particulate matter, such as fat globules and casein micelles from the milk feedstream. The concentration/fractionation steps remove most small molecules, including lactose, minerals and water, to increased purity and reduce volume of the product. The product of the TFF process is thereafter concentrated to a level suitable for optimal downstream purification and overall product stability. This concentrated product, containing the molecules of interest, is then aseptically filtered to assure minimal bioburden and enhance the stability of the molecules of interest for extended periods of time. According to a preferred embodiment of the current invention, the bulk product will realize a purity between 65% and 85% and may contain components such as goat antibodies, whey proteins (β Lactoglobulin, α Lactalbumin, and BSA), as well as low levels of residual fat and casein. This partially purified product is an ideal starting feed material for conventional downstream chromatographic techniques to further select and isolate the molecules of interest which could include, without limitation, a recombinant protein produced in the milk, an immunoglobulin produced in the milk, or a fusion protein.

Step # 1 (Clarification)

Turning to FIG. 5, transgenic mammal milk, preferably of caprine or bovine origin, is clarified utilizing batch-wise microfiltration. The milk is placed into a feed tank and pumped in a loop to concentrate the milk retentate two fold (see flow diagram in FIG. 5). Once concentrated the milk retentate is then diafiltered allowing the product and small molecular weight proteins, sugars, and minerals to pass through an appropriately sized membrane. According to the current invention, this operation is currently designed to take 2 to 3 hours and is will process 1000 liters of milk per day. The techniques and methods of the current invention can be scaled up and the overall volume of product that can be produced is dependent upon the commercial and/or therapeutic needs for a specific molecule of interest.

Step # 2 (Concentration/Fractionation)

Again referring to FIG. 5, the clarified permeate from the first step is concentrated and fractionated using ultrafiltration (“UF”). The clarified permeate flows into the UF feed tank and is pumped in a loop to concentrated the product two-fold. Once the concentration step is initiated the permeate from the UF is placed into the milk retentate in the clarification feed tank in the first step. The first and second step are sized and timed to be processed simultaneously. The permeate from the UF contains small molecular weight proteins, sugars, and minerals that pass through the membrane. Once 95% of the product is accumulated in the retentate of the UF, the clarification is stopped and a concentration/diafiltration of the UF material is begun. The product is concentrated 5 to 10 fold the initial milk volume and buffer is added to the UF feed tank. This washes away the majority of the small molecular weight proteins, sugars, and minerals. This operation is currently designed to take 2.5 to 3.5 hours and can process up to 500 liters of clarified permeate per day. As above, the techniques and methods of the current invention can be scaled up and the overall volume of product that can be produced is dependent via this concentration/fractionation process is dependent upon the commercial and/or therapeutic needs for a specific molecule of interest.

Step # 3 (Aseptic Filtration)

According to FIG. 5, and according to the current invention, the clarified bulk concentrate is then aseptically microfiltered. The resulting 50 to 100 liters of UF retentate is placed into a feed tank where it is pumped through a dead-end absolute 0.2 μm MF filtering system in order to remove the majority of the bioburden and enhance stability of the product for extended periods of time. The product is pumped through the filtering system of the invention and may then be directly filled into a final packaging configuration. Under conditions for processing a molecule of interest in a GMP facilities meeting clean room specifications (e.g., class 100 conditions) This operation is currently designed to take 0.5 to 1 hour and will process up to 100 liters of clarified bulk intermediate per day. As above, the techniques and methods of the current invention can be scaled up and the overall volume of product that can be produced is dependent via this concentration/fractionation process is dependent upon the commercial and/or therapeutic needs for a specific molecule of interest.

Consistency of Biochemical Properties Between rhAT and Human Plasma Derived Antithrombin

Antithrombin is a 58,000 dalton serine protease inhibitor of the serpin type that is the principal inhibitor of the blood coagulation serine proteases thrombin and Factor Xa, and to a lesser extent, factors IXa, XIa, XIIa, trypsin, plasmin, and kallikrein (Aubry 1972, Menache 1991, Menache 1992). The rhAT of the current invention neutralizes the activity of thrombin as well as other serine proteases by forming a 1:1 stoichiometric complex between the active serine residue on the proteinase and the active site arginine of the inhibitor.

In vivo, antithrombin (“AT”) is synthesized in the liver and is present in humans at serum at levels of 12.5 mg/dl to 15 mg/dl (Murano 1980). A small fraction of the circulating AT is normally bound to proteoglycans on the surface of vascular endothelial cells. These proteoglycans are predominantly heparan sulfate, a molecule structurally similar to heparin, which is able to catalyze the inhibition of thrombin in the same way as heparin. This binding catalyzes a 1000 fold increase of AT inhibitory activity toward thrombin and Factor Xa. This localization of a fraction of the AT on the endothelial surface, where enzymes of the intrinsic coagulation cascade are commonly generated, enables AT to rapidly neutralize these hemostatic enzymes and protect natural surfaces against thrombus formation (Rosenberg, 1989).

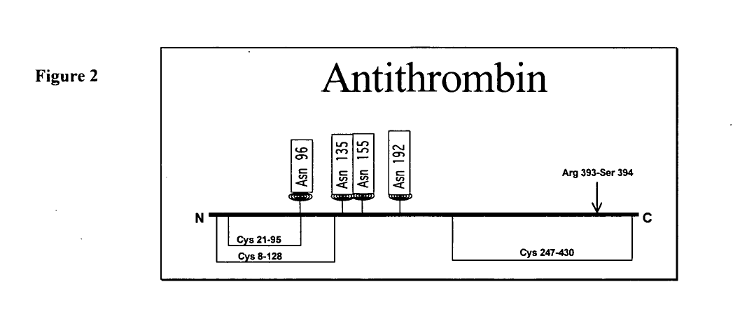

Human plasma-derived AT (“hpAT”) contains 432 amino acids and has a carbohydrate content of about 15% (Franzen 1980) with a total MW of 58 kD. The protein (FIG. 2) has three disulfide bridges connecting Cys8-128, Cys21-95, and Cys247-430 and four carbohydrate side chains located at Asn96, Asn135, Asn155 and Asn192 (Petersen 1979, Pratt 1991). The type of oligosaccharide found on the hpAT is consistent on all four sites and is mainly comprised of fully sialylated, non-fucosylated, and biantennary complex oligosaccharides (Mizuochi 1980). Recombinant human AT isolated from the milk of transgenics goats (engineered to specifically express human AT in their mammary gland) is comparable to human plasma derived AT (Table 1) with respect to purity, activity, degree of oxidation, primary sequence, secondary and tertiary structure (Edmunds et al 1998; Van Patten et al. 1999). A difference observed between hpAT and rhAT is the glycosylation pattern which leads to a 4 fold (4×) higher affinity of rhAT for heparin. This difference does not affect the primary function of rhAT, which is inhibition of thrombin or Factor Xa in the presence of excess heparin moieties. This inhibitory function is the primary mode of action of AT in the hereditary deficiency (HD) indication where HD patients are at risk of venous thrombosis in high-risk situations such as surgery or pregnancy (reviewed in Van Boven & Lane, 1997; Schinzel & Weilemann, 1998).

Table 1 a summarizes the biological and physical consistency of rhAT and commercial preparations of hpAT licensed for use in treatment of subjects who are congenitally AT deficient. The sections that follow provide additional details on these comparative parameters. Most of the side-by-side biochemical comparison studies that follow used Bayer's Thrombate as a comparator, since it is the only hpAT that is commercially available in the US. A few studies used as the comparator Behringwerke's Kybernin (now Aventis Behring) that is only available in Europe. The identity of the plasma derived AT comparator will be noted for each study. In some cases direct head to head comparisons were not made and the comparator will be published literature values for hpATs licensed in the US (Bayer-Thrombate) and EU (Grifols-Anbin, Pharmacia-ATnativ, Immuno-Antithrombin Immuno, Aventis Behring-Kybernin).

TABLE 1a
Consistency of hpAT and rhAT Products for Use as a
Biopharmaceutical

| hpAT | rhAT |
|---|---|
| Single chain of 432 amino acids | Identical single chain of 432 amino acids |
| 6 cysteine residues forming 3 disulfide bonds | Same 6 cysteine residues forming 3 |
| (Cys 21-95, Cys 8-128, Cys 247-430) | disulfide bonds (Cys 21-95, Cys 8-128, |
| | Cys 247-430) |
| Serine protease inhibitor (serpin) | Serine protease inhibitor (serpin) |
| Contains the reactive center Arg 393-Ser 394 | Contains the reactive center Arg 393-Ser |
| which provides a cleavage site for | 394 which provides a cleavage site for |
| proteinases such as thrombin | proteinases such as thrombin |
| Inhibits thrombin and Factor Xa in in vitro | Inhibits thrombin and Factor Xa in in |
| inhibition assay | vitro inhibition assay |
| Binds to heparin, which catalyzes a | Binds to heparin, which catalyzes a |
| conformation change and an increase in | conformation change and an increase in |
| activity | activity. |
| Distinctive far and near UV circular | Same near and far CD profile |
| dichroism (CD) profile | |
| Specific activity of US licensed hpAT | Specific activity of rhAT product ˜7 IU/mg |
| product (Bayer's Thrombate) ˜7 IU/mg, EU | |
| licensed products range from 3.9-6.6 IU/mg | |
| >95-99% pure depending on manufacturer | >99% pure* |
| Low level of methionine oxidation | Level of methionine oxidation |
| (Thrombate) | comparable to Thrombate. |
| 4 N-linked glycosylation sites (Asn 96, 135, | 4 N-linked glycosylation sites (Asn 96, |
| 155, 192) | 135, 155, 192) |
| 85-95% alpha isoform with 4 biantennary, | Biantennary, mono and disialylated |
| mono and di-sialylated oligosaccharide | oligosaccharide chains at three sites with |
| chains | hybrid and high mannose structures at |
| 5-15% is the high heparin affinity beta | Asn 155, |
| isoform lacking glycosylation at Asn 135 | Substitution of some N-acetyl neuraminic |
| 2-6 terminal sialic acid linkage | acid with N-glycolyl-neuraminic acid |
| | Also contains the beta isoform lacking |
| | glycosylation at Asn 135 (≦20%) |
| | 2-6 terminal sialic acid linkage |
| Recognized by polyclonal and monoclonal | Recognized by same polyclonal and |
| antibodies | monoclonal antibodies |
| 2-3 yr shelf life depending on manufacturer | Proposed 2-3 yr shelf life. |

*(Depends on methodology used. Heat causes aggregates and therefore somewhat lower molecular weight bands. The figures here relate these as product monomers.)

Purity of rhAT

In comparing the purity of rhAT to that of commercially available hpAT, the principal considerations are related to the differences in origin of the source material, i.e. goat milk versus human blood and thus the contaminating protein profile. The other potential contaminant in commercial hpAT is heparin, which leaches off the heparin affinity columns used to purify all AT preparations.

With Regard to Contaminating Goat Proteins (Including Endogenous Goat AT)

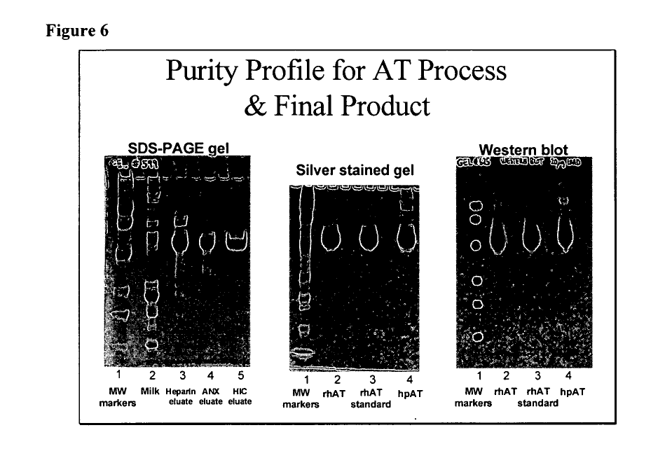

The purity of rhAT was greater than 99% after the three chromatographic steps and was at least equivalent to that observed for thrombate as judged by SDS-PAGE (Edmunds et al. 1998). Thrombate is reported to be >95% pure with >95% active hpAT. No protein bands other than AT were evident with silver staining at high protein loads (FIG. 6). The higher molecular weight bands seen with both rh and plasma human AT are aggregates of AT. Published purity values for EU licensed hpATs range from >95 to 99% pure. Looking at FIG. 8, using reverse phase-HPLC analysis, the purity of the rhAT was confirmed to be greater than 99%. The chromatographic profile and retention time of the rhAT was similar to the hpAT (Thrombate & Kybernin). Analysis of the leading shoulder areas, similarly seen in both rhAT and hpAT chromatograms, by peptide mapping coupled with mass spectrometry, identified these peaks as AT molecules that are partially oxidized (Edmunds et al. 1998). Five methods have been developed, validated and implemented to detect potential milk contaminants in rhAT drug substance. Characterization studies have determined the immunoreactivity, specificity and limits of quantitation for each of the assays (Table 1b).

TABLE 1b
Methods to Detect Residual Goat Milk Proteins in rhAT Drug Substance

| | | Specification |
|---|---|---|
| Assay | Detection | (ng/mg) |
| Soluble Goat | Quantitative method for whey | ≦1 |
| Milk Protein | proteins α-lactalbumin, albumin, β- | |
| (SGMP) ELISA | lactoglobulin | |
| | LOQ is 5 ng/mL or 0.2 ng/mg. | |
| Colloidal Goat | Quantitative method for casein: α S1- | ≦0.9^{a} |
| Milk Protein | casein, α S2-casein, β-casein, κ- | |
| (CGMP) ELISA | casein | |
| | LOQ is 20 ng/mL or 0.9 ng/mg. | |
| Goat IgG ELISA | Quantitative method specific for goat | ≦0.3^{a} |
| | IgG, with some cross-reactivity for | |
| | IgA and IgM | |
| | LOQ is 6 ng/mL or 0.3 ng/mg. | |
| Immunological | Quantitative method for residual | ≦0.3 |
| Detection of | lactoferrin and lactoperoxidase | |
| Contaminating | LOQ is 0.625 ng/mL or 0.03 ng/mg | |
| Protein (IDCP) | | |
| ELISA | | |
| Goat Antithrombin | Limit test for goat antithrombin | ≦2.5^{a} |
| (gAT) Western | LOD is 2.5 ng/mg. | |

^{a}Quantitation limit of the method

Using validated methods the amount of contaminating goat proteins has been measured in bulk batches of rhAT. RhAT is very pure and contains not more than 5 ng of contaminating goat proteins per mg of rhAT.

There is approximately 85% sequence homology between human and goat plasma AT. Endogenous goat AT is present in goat milk, as are other goat serum proteins, but at a lower level than found in serum (1/50 to 1/100 of that in serum). It is not unexpected therefore, that small amounts of endogenous goat AT contaminate the recombinant human AT expressed in the milk of transgenic goats. Goat AT from non-transgenic normal goat milk was partially purified by Heparin affinity chromatography and the goat AT quantitated by reversed phase HPLC. The goat AT concentration in the partially purified preparation was approximately 5.6 mg/L. The average concentration of rhAT in the transgenic animals used to produce the clinical materials is 2.2 g/l. This gives a calculated ratio of 2.5 mg of goat AT per g of rhAT in transgenic goat milk.

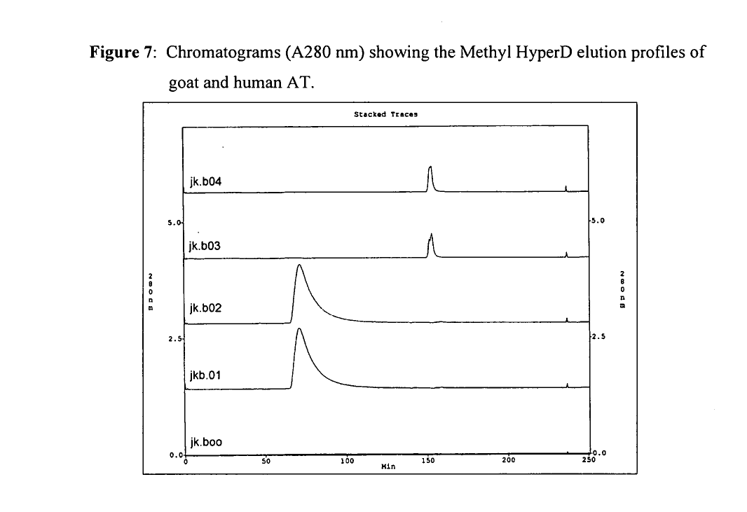

Although very little goat AT is present in milk (5.6 mg/L), it does co-purify with the rhAT on the heparin column. To reduce the level of endogenous goat AT in the purified rhAT, a hydrophobic interaction chromatographic (HIC) separation step using Methyl HyperD (BioSepra, Marlboro, Mass.) was incorporated into the purification process. This step exploits a minor difference in the hydrophobicity between the goat and human antithrombin proteins. A small-scale validation study was completed. In side by side experiments, goat AT and rhAT were separated under identical conditions on appropriately scaled-down columns. For this study, measurements of goat or transgenic human antithrombin levels were made by quantitative reverse phase analytical methods. Baseline separation of goat and human AT were seen in this study (FIG. 7).

The goat AT reduction obtained for the duplicate runs was calculated to be 3.15 and 3.17 Log10. The ratio of goat AT to rhAT in the starting transgenic goat milk was estimated to be 2.5 mg/g of AT. Using this data, the theoretical level of goat antithrombin contamination in the final product can be determined to be 1.6 ng/mg of AT. (2,500/10^{3.2}=1.6 ng of goat AT/mg of rhAT). Residual goat plasma AT levels in the formulated bulk batches (25 mg/ml) were also directly measured by a validated specific goat AT ELISA. The contaminating goat AT levels were <12.5 ng/mL, which is the limit of detection of this assay.

Residual Heparin Contamination

As mentioned previously, a heparin column is used in the purification process for hpAT and several EU available AT products contain measurable residual heparin. Heparin levels have been reported (Hellstern et al., 1995) in 5 commercial hpAT concentrates available in the EU from 4 manufacturers (Table 2).

TABLE 2

| | | | | Pharmacia | |
|---|---|---|---|---|---|
| | Alpha | Behring | Immuno | (current) | Pharmacia (new) |
| Heparin | 0.6 (0-1.0) | 1.6 (1.5-2.0) | 1.9 (0-6.5) | 1.2 (1.0-1.5) | 0.9 (0.5-1.0) |
| (IU/vial) | | | | | |

Numbers in parenthesis indicate the range detected in samples from multiple vials.

Residual heparin can be problematic for certain patients with heparin-induced thrombocytopenia. Oozing and hematoma can be seen as side effects in preparations of AT concentrate that contained excess heparin. The FDA approved product has a heparin specification of ≦0.004 U heparin/IU hp AT. Therefore, a limit assay for heparin in the rhAT final product has been developed and used in comparative analysis with Thrombate. This assay is used clinically to measure plasma heparin levels in patient samples and utilizes Factor Xa as a substrate.

The limit of detection of the assay is 0.004 units heparin/unit of AT. In side-by-side tests, no heparin was detected in the rhAT purified by the methods of the instant invention or in Thrombate. Given the limit of detection of the method, only results less than 0.004 units heparin/unit AT can be reported. Both heat-treated and non-heat treated rhAT samples have been tested with identical results.

rhAT Physical Structure

Amino Acid Sequence

The rhAT of the instant invention is made from the milk of transgenic animals. Though it could be sourced from various transgenic animal sources it was made for the experiments of the current invention from the milk of transgenic goats. The Thrombate used for comparison studies herein was isolated from pooled human plasma and contains the same 432 amino acids as determined by amino terminal sequence analysis, peptide mapping, and LC/MS analysis (Edmunds et al. 1998). N-terminal sequence analysis confirmed that the rhAT had the correct N-terminal sequence. The reduced and pyridylethylated peptide map of rhAT was essentially identical to that of Thrombate. The only differences noted were in the regions of the three glycopeptides due to the glycosylation heterogeneity in the rhAT. The primary sequence of rhAT was confirmed by on-line LC/MS analysis of an endoproteinase Lys-C digest.

AT contains four methionine residues, which may be prone to oxidation under forced conditions in vitro. Normally, there is low oxidation of AT. In a comparative study of rhAT, Thrombate and Kybernin, all three AT preparations were found to have similar low levels of methionine oxidation (FIG. 8) (Van Patten et al., 1999). It was also demonstrated that methionine oxidation had little impact on the inhibitory activity of rh or hp AT.

Secondary Structure

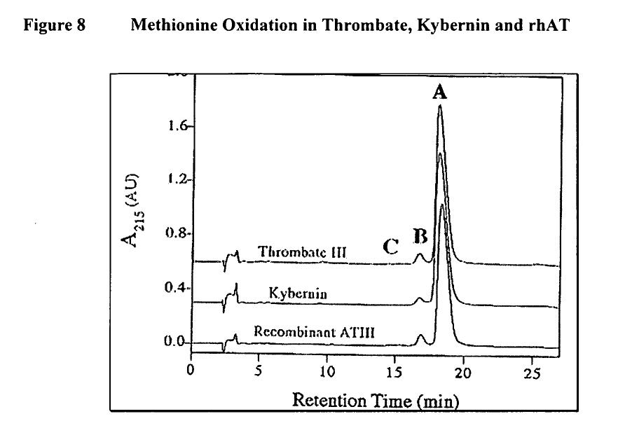

Both rhAT and hpAT contain the same three disulfide bonds (Cys 8-128, Cys 21-95, Cys 247-430) (FIG. 9) as determined by peptide mapping under non-reducing conditions (Edmunds et al. 1998). The conformation of rhAT was analyzed further by CD spectroscopy (Edmunds et. al 1989). Far UV spectra are sensitive to alterations in secondary structure and near UV spectra are sensitive to alterations in tertiary structure. The far UV CD spectrum was similar for both proteins and was characterized by two negative bands and a positive maximum indicative of the presence of both α-helix and β-sheet in the protein, which was consistent with the crystal structure data of hpAT (Schreuder et. al, 1994). In the far UV CD, the addition of heparin produced little change, which suggested that secondary structures were not altered.

The near UV spectra of both proteins were also similar (Edmunds et al. 1998) and in excellent agreement with previously published spectra of AT derived from human and bovine plasma (Nordenman et al 1977). The near UV spectrum showed a dramatic increase in band intensity across the whole region when heparin was added to both proteins. This increase was attributable to the conformational change of buried and exposed tryptophan residues upon heparin binding.

Glycosylation Analysis

By on-line LC/MS analysis of an endoproteinase Lys-C digest, the only post-translational modifications detected were at the known N-glycosylation sites on either rhAT or hpAT (Edmunds et al. 1998). Both rhAT and hpAT (Thrombate) contain the same 4 N-linked glycosylation sites (Asn 96, 135, 155, 192) as determined by peptide mapping and by LC/MS. No evidence of O-linked glycosylation was observed during LC/MS analysis of both proteins. Human plasma AT lacks glycosylation at the Asn 135 (the β-isoform) in 5% to 15% of the total AT found in plasma (Turk et al, 1997; Swedenborg 1998). LC/MS data indicated that the rhAT had glycosylation at Asn 135 greater than 80% of the time.

As inferred previously from peptide maps, the monosaccharide composition of rhAT was different from that of Thrombate (Edmunds et al. 1998). HpAT has predominantly identical oligosaccharides on the 4 N-linked glycosylation sites (Franzen & Svensson, 1980; Mizuochi et al., 1980), although between 15 to 30% of the chains may lack terminal sialic acid (Fan et. al. 1993; Zettlmeissl et al. 1989). The main glycosylation differences observed for the rhAT were the presence of fucose and GalNAc, a higher level of mannose and a lower level of galactose and sialic acid. There is also substitution of 35-55% of the N-acetyl neuraminic acid with N-glycolyl-neuraminic acid. As expected from the monosaccharide compositional analysis, the LC/MS analysis was more complex for all the rhAT glycopeptides than for hpAT. The terminal sialic acid in the rhAT contains the same 2-6 linkage found in hpAT (Munzert et al 1996; Fan et al 1993). Several laboratories have determined that differences in glycosylation of AT do not affect the intrinsic rate constant of the uncatalyzed or heparin catalyzed inhibition of thrombin indicating that the carbohydrate chains solely affect heparin binding and not heparin activation or proteinase binding functions (Bjork et al 1992, Erdsal-Badju et al 1995 and Olson et al 1997). Thus, glycosylation differences do not impact the major biological activity of AT which is thrombin inhibition.

In Vitro Response

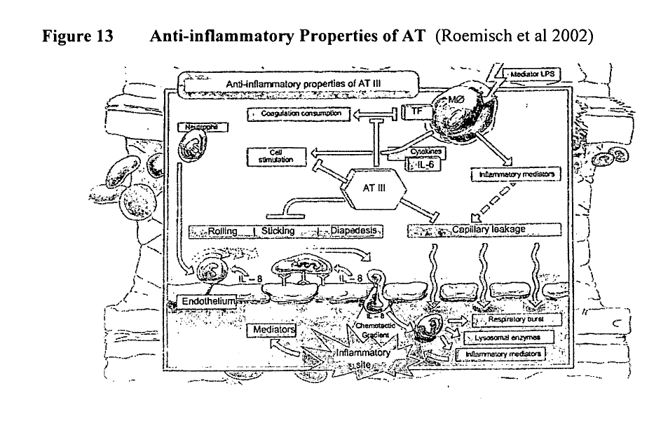

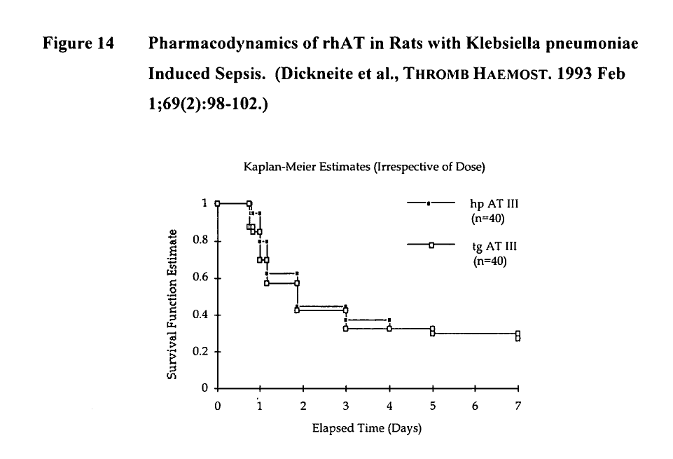

As mentioned previously, AT has significant anti-inflammatory properties (FIG. 13) (reviewed in Roemisch et al, 2002). Several collaborators have used rhAT in comparative studies with hpAT in various in vitro cell based assay systems.

Syndecan-4 as Antithrombin Receptor of Human Neutrophils

AT inhibits chemokine-induced migration of neutrophils by activating heparan sulfate proteoglycan (HSPG)-dependent signaling (Kaneider et al, 2001). The object of this study was to characterize the mechanism by which AT regulates migration of neutrophils, which are involved in a variety of conditions including inflammatory diseases. Human neutrophils were obtained from healthy volunteers and migration was measured in modified Boyden chambers. Either Kybernin P or rhAT was used as an attractant. RhAT was at least as effective in deactivating neutrophil chemotaxis as the Kybernin P.

To investigate the role of intact HSPG on the neutrophil surface for AT-induced cell migration, neutrophils were pretreated with heparinase or chondroitinase. Chemotactic effects of hpAT (1 U/mL) or rhAT (1 U/mL) were completely abolished by pretreatment with both agents, suggesting that syndecans mediate direct cellular actions of AT. Antibodies to syndecan-4 also inhibited rhAT-induced migration of neutrophils. Collectively, the data suggests that AT regulates neutrophil migration via effects of its heparin-binding site on cell surface syndecan-4. Aspects of this work with rhAT, confirm previous studies of binding of hpAT to syndecans on the cell surface and AT inhibition of neutrophil chemotaxis (cited in Kaneider et al 2001).

In a follow-up study, Kaneider et al (2002) showed that all AT concentrates irrespective of the pharmaceutical source deactivated neutrophil chemotaxis toward IL-8. However, differences in response level were seen depending on the source of commercial AT, suggesting that at equivalent WHO standard concentrations, clinical AT concentrates may differ in anti-inflammatory potential.

Biological Consistency of rhAT and hpAT in Animal Sepsis Models Comparison of the Effects of hpAT and rhAT on the Survival of Rats with Klebsiella pneumoniae Induced Sepsis

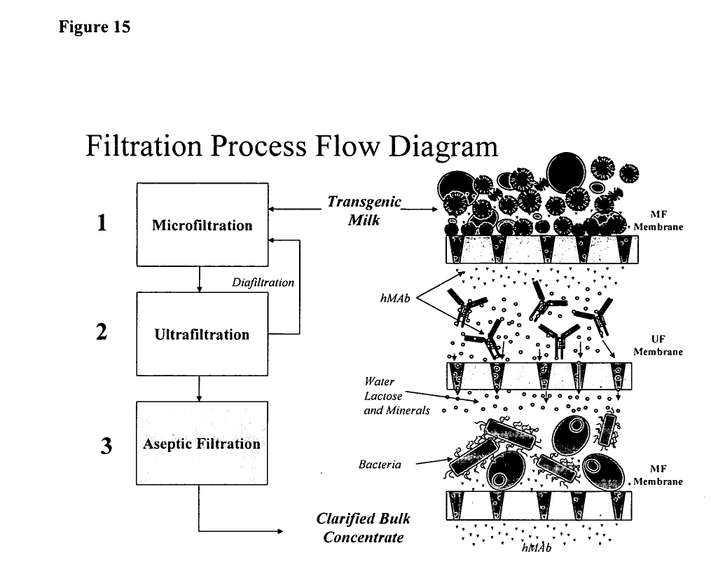

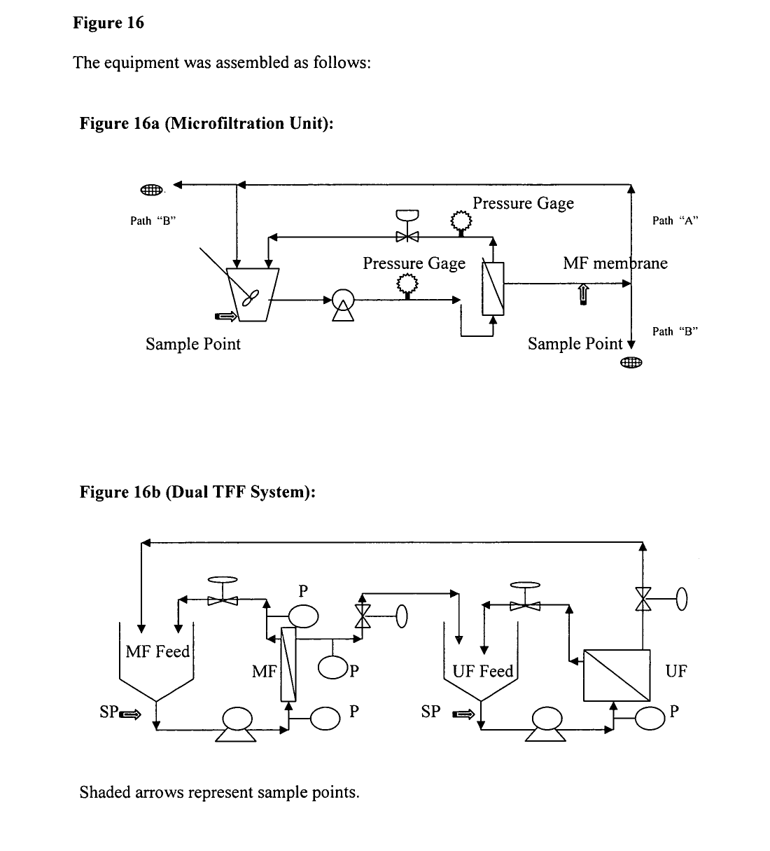

This study was performed at Behringwerke AG under the direction of Dr. G. Dickneite. Four groups of 20 rats each were administered 3 doses of either rhAT (250 or 500 U/kg) or hpAT (250 or 500 U/kg). Doses were administered intravenous at 3, 19 and 48 hours following the induction of sepsis by an intravenous injection of Klebsiella pneumoniae (8×107 cfu). Tobramycin was administered 1 hour after induction of sepsis (at 2 mg/kg) to control infection. In this model, the hazard ratio was for rhAT compared to hpAT was 1.1 (FIG. 15). Statistically, rhAT was as effective as hpAT in promoting survival. (Dickneite G et al, THROMB HAEMOST. 1993 February; 69(2):98-102).

Evaluation of rhAT in Baboon Sepsis Model (Minnema et al, 1999)

This study was performed under the direction of Dr. F. Taylor. The objective of this study was to evaluate the protective effect of rhAT against bacterial sepsis in baboons. Five adult animals were used. The dosing regimen involved administration of 500 U/kg rhAT as 0.5 hr infusions at t=−1 hr and at t=+3 hrs and 250 U/kg rhAT as a bolus at the time of E. coli challenge (t=0 hr).

Administration of rhAT protected three of the five baboons from a lethal dose of E. coli. One of the five animals that died (baboon #2) was not administered the third dose of rhAT. The cause of death of this animal (which occurred at t=28 hrs) was sepsis. The cause of death of baboon #4 was attributed to capillary leakage in the lungs consistent with adult respiratory distress syndrome (ARDS). Death occurred at t=57 hours. There was no evidence of disseminated intravascular coagulation (DIC). These results indicate that, when given in the appropriate doses, rhAT protects against DIC and, in 3 of 4 cases, rhAT protects against death from a lethal dose of E. coli.

In this study, rhAT established an improved survival and protected against diffuse intravascular coagulation (DIC) (Minnema et al, 2000). In published studies using the same baboon model in the same laboratory, it was reported that hpAT also promoted survival and protected against DIC (Taylor et al., 1988). The rhAT group had an accelerated increase of thrombin-AT complexes and significantly less fibrinogen consumption as compared to control non-treated animals. The protective effect of rhAT on fibrinogen consumption was similar to that reported for hpAT (Taylor et al., 1988) and consistent with the ability of rhAT to prevent DIC. The rhAT group had much less severe thrombotic pathway on autopsy and virtually no fibrinolytic response to E coli challenge. There was a marked inhibition of the sepsis-induced elevation of tPA in these animals and PAP complexes were not formed. Additionally, the rhAT group had a significantly attenuated inflammatory response with a marked reduction of cytokine release. IL-10, IL-6 and IL-8 concentrations were significantly lower in the rhAT treated animals. The inhibitions of IL-6 and IL-8 have also been seen with hpAT in other sepsis models.

Antithrombin III Reduces Mesenteric Venule Leukocyte Adhesion and Small Intestine Injury in Endotoxemic Rats (Neviere et al, 2001)

AT has also been shown to effect leukocyte adhesion possibly by effecting prostacyclin production. To test whether the rhAT molecule had similar properties, rhAT was studied in a leukocyte adhesion model. The effect of rhAT on leukocyte adhesion was examined by measuring rolling and firm adhesion of leukocytes in mesenteric venules of endotoxemic rats using intravital microscopy (Neviere et al, 2001). Endotoxemia was induced by the administration of 10 mg/kg of endotoxin, intravenously. Then rats were treated either with saline or rhAT (250 and 500 U/kg). Following anesthesia, the distal ileum was exteriorized and the mesentery was inserted in an intravital microscopy chamber. Mesenteric circulation was observed with the use of an intravital microscope fitted with a video camera system. Leukocyte rolling and adhesion in the mesenteric venules were monitored. Flux of rolling leukocytes was measured as the number of white blood cells that could be seen rolling past a fixed perpendicular line in the venule during a 1-minute interval. Quantification of venular endothelium leukocyte adherence was performed off-line by playing back videotaped images and counting the number of leukocytes that stuck and remained stationary for a period >30 s. rhAT (250 U/kg and 500 U/kg) was shown to attenuate both endotoxin-induced venular leukocyte rolling and adhesion in a dose-dependent manner. Pretreatment with indomethacin, a prostaglandin synthesis inhibitor completely abolished the effect on leukocytes rolling and adhesion, suggesting that the effect of AT could be mediated by an effect on prostacyclin production.

This effect on leukocyte adhesion obtained with rhAT is similar to the activity of hpAT observed in related models. For example, in skinfold of endotoxemic Syrian hamster, multiple injections of 250 U/kg of hpAT attenuated LPS-induced arteriolar and venular leukocyte adhesion (Hoffman et al 2000, Hoffman et al 2002). Here again this effect was completely abolished by pretreatment with indomethacin. Previously, in a feline mesentery ischemia/reperfusion using intravital microscopy to monitor leukocyte rolling and adhesion, pretreatment with hpAT (250 U/kg) reduced neutrophil rolling and adhesion to preischemic levels during reperfusion (Ostrovsky et al 1997).

Effects of Combination Therapy on Disseminated Intravascular Coagulation

Disseminated Intravascular Coagulation (DIC) is the ultimate hemostatic imbalance between coagulation and anticoagulation systems. This devastating disease is a combination of uncontrollable bleeding and excessive clotting precipitated by vascular injury, acidosis, endotoxin release and sepsis. This phenomenon is commonly seen in sick neonates who have innately lowered levels of coagulation factors including plasminogen, AT and protein C. By far, the most common cause of DIC is sepsis, with an incidence of one to five per 1,000 live births and a mortality rate of 15-50%. For this study, the working hypothesis was that attacking the fibrinolytic as well as the anticoagulant derangement in a newborn piglet model should improve therapeutic efficacy. RhAT replacement should replenish diminished anticoagulation factors thus decreasing clot formation. AT blocks microthrombus formation by binding and inactivating thrombin and Factor Xa. R-TPA supplementation will affect the defective fibrinolytic pathway initiating fibrinolysis of existing microthrombi by activating plasmin to cleave fibrin and fibrinogen.

DIC was induced in neonatal pigs (7-20 days old) by giving them 800 micrograms/kg of E. Coli LPS over 30 min. The pigs were divided into 4 groups. Group A had LPS alone-supported with fluids and pressors (dopamine and dobutamine), Group B had LPS followed by rhAT administration with support from fluids, pressors and additional rhAT, Group C had rTPA alone as treatment after LPS-supported by fluids, pressors and additional rTPA and Group D had rTPA and rhAT as treatment after LPS-supported by fluids, pressors and additional rTPA and rhAT. The four groups were monitored for 7 hours with periodic hematologic and coagulation studies (Table 24). Surviving pigs were euthanized and their organs examined grossly and microscopically.

TABLE 24
Hematologic and Coagulation Results

| | Survival | Platelet | Fibrinogen |
|---|---|---|---|
| | time | decrease | decrease |
| Group | (hrs) | by 4 hrs | by 4 hrs |
| A | 0.5-5.5 | 36-91% | 43-77% |
| B (rhAT) | 6-7 | 35-56% | 2% |
| C (rTPA) | 0.5-7 | 43-75% | 55-70% |
| D (rhAT + rTPA) | 7 | 46% | 14% |

The longest surviving animals were those that received rhAT either alone or in combination with rTPA. In the untreated controls there were hemorrhages in the kidneys, liver, lungs and heart. In the pigs receiving rhAT alone or in combination with rTPA, minimal to no lung hemorrhage was observed. The investigators concluded that rhAT and rTPA decreased the drop in platelets and fibrinogen thus inadvertently decreasing the risk of bleeding. Maintaining higher levels of AT and giving TPA continuously works on forming and existing clots to attack the coagulation aspect of DIC. The decreases in drops in platelets and fibrinogen have also been observed with hpAT in a variety of sepsis animal models.

RhAT in Human Clinical Studies

RhAT has been successfully used in seven clinical studies (Table 25) to determine its efficacy in the heparin resistance indication for patients undergoing cardiac surgery involving CPB or for repletion of normal AT levels in patients who have a hereditary deficiency of AT and who are in high risk situations such as delivery or surgery. In all the human studies completed to date, rhAT has proved safe and met the primary endpoints of that study. Although only one study, which was aborted before completion for non-safety reasons (AT97-0903), involved a head to head comparison of rhAT and hpAT, several studies (AT96-0801, AT97-0502, AT97-0504 and AT97-0903) provide information that can be compared in some way to published reports on hpAT bioactivity in similar circumstances. Only trial design information and data corresponding to clinical measures of anticoagulant and fibrinolytic activity will be discussed below. Immunological safety data was discussed earlier. All studies demonstrated that rhAT was well tolerated and safe in these patient populations.

TABLE 25
Use of rhAT in Human Subjects

| | | Phase 1, |
|---|---|---|
| Gen/G-9691 | Normal volunteers | single ascending dose PK |
| AT96-0801 | CPB patients | Phase II, single escalating dose |
| AT97-0502 | CPB patients (heparin | Phase III, double-blind, placebo |
| | resistant) | controlled |
| AT97-0504 | CPB patients (heparin | Phase III, double-blind, placebo |
| | resistant) | controlled |
| AT97-0903 | CPB patients | Double blind, randomized rhAT |
| | | vs hpAT |
| AT III-006-00 | Normal volunteers | Single dose, randomized, open |
| | | label, rhAT cross-over study |
| ATIII-009-00 | Patients with AT HD | Phase I, single dose PK study |
| | Patients with AT HD | Phase III, efficacy study |
| Compassionate | Patients with AT | Applicable during periods where |
| use cases | deficiency | Thrombate is not available in the |
| | | US. |

AT96-0801 Published as “Recombinant Human Transgenic Antithrombin in Cardiac Surgery: A Dose-Finding Study” (Levy et al, 2002).

Acquired AT deficiency may render heparin less effective during cardiac surgery and CPB. The study was designed to examine the pharmacodymanics and optimal dose of rhAT need to maintain normal AT activity during CPB, optimize the anticoagulant response to heparin and attenuate excessive activation of the hemostatic system in patients undergoing coronary artery bypass grafting (CABG). During CPB, AT activity frequently decreases as low as 30-50% of normal. Low AT concentrations during cardiac surgery are likely to develop because of the preoperative use of heparin, the effect of hemodilution on the pump, and CPB-associated excessive hemostatic system activation. Anticoagulation is used during cardiac surgery to prevent thrombosis of the extracorporeal circuit and to minimize CPB-related activation of the hemostatic system. In some cases when heparin alone is not effective, either fresh frozen plasma or hpAT concentrates have been used in patients that show an appreciable heparin resistance prior to initiation of CPB. However AT concentrate has not been approved for this indication in the US.

A single center, open-label, single dose, dose escalation study (GTC AT 96-0801) was conducted in 36 patients, between the ages of 18-80 years, admitted for primary cardiac surgery requiring CPB. All patients underwent elective primary CABG and had been on heparin therapy at least 12 hours prior to surgery. Thirty patients received rhAT and 6 patients received placebo. Patients receiving active drug were divided into groups of 3 and assigned to one of 9 dosing cohorts. The individual treatment dosing cohorts were 10, 25, 50, 75, 100, 125, 150, 175, and 200 U/kg rh AT. A tenth placebo cohort was added which included an additional 3 patients.

No patients developed circulating antibodies to rhAT following treatment, two patients had no post-drug samples taken. Supplementation of rhAT significantly (P<0.0001) improved heparin responsiveness as measured by an increase in the ACT (844±191 s) as compared to heparin administration alone (531±180 s). Furthermore, AT supplementation resulted in significantly (P=0.001) better inhibition of thrombin (as measured by a decrease in fibrin monomer) and fibrinolysis (as measured by a decrease in D-dimer) at doses up to 125 U/kg. There was also a reduced impairment of platelet function after CPB, which is thought to be the most important hemostatic defect after CPB. Results suggest that single rhAT doses of 75 U/kg and higher will maintain the AT activity level at greater than 100% throughout the course of CPB. Although this study did not include a direct comparison with hpAT it does support the observation that the rhAT and hpAT are biologically consistent and are reflective of results that would be expected if hpAT had been administered.

AT97-0903

The original purpose of this study was to evaluate and compare the safety and efficacy of 15 U/kg and 75 U/kg rhAT with 15 U/kg human plasma derived AT (hpAT) in restoring heparin sensitivity to heparin resistant patients undergoing cardiac surgery requiring cardiopulmonary bypass. This study was conducted in approximately 18 USA and European centers and was originally designed to enroll approximately 378 patients. The primary objective was later revised in a protocol amendment to compare the difference in the ability of a high dose (75 u/kg) and a low dose (15 u/kg) of rhAT to restore the ACT response to heparin in heparin resistant patients, thus allowing them to successfully proceed on to CPB and surgery. To accommodate this objective, the sample size was reduced to 270 patients.

As a result of subsequent discussions with several regulatory authorities, the study was terminated in May 1999. At the time of study termination, a total of 47 patients had been entered into the study. By direct comparison of rhAT to hp AT, this study demonstrates that both rhAT and hpAT restore the anticoagulation responses in the clinical setting. In those patients studied, AT activity levels in each treatment group were increased from baseline values that were equivalent. Importantly, the change from baseline (increase) observed in the 15 U/kg hpAT group (n=14) and the 15 U/kg rhAT group (n=15) was comparable and did not differ significantly during the treatment period. However, as expected, the 75 U/kg rhAT group (n=18) experienced a change from baseline (increase) that was significantly greater than the change (increase) observed in the 15 U/kg hpAT group or 15 U/kg rhAT group during the treatment period. In this comparative trial with hp AT, rhAT had the same biological effect as measured by change in AT activity.

Although limited, the comparative safety data provided by this study further supports biological consistency of rhAT and hp AT. Intravenous administration of 15 U/kg or 75 U/kg rhAT appear to have similar safety profiles when compared to each other, and when compared to a 15 U/kg hpAT control group.

Compassionate Use in Hereditary AT Deficient Patient Cases (Konkle et al, 2003)

Hereditary AT deficiency is associated with a significant risk of venous thrombosis in high risk situations such as delivery and surgery. HpAT concentrate (Bayer-Thrombate) has been approved in the US for replacement when anticoagulation is interrupted in these patients. However, Thrombate supplies have been limited and there are periods when it is not available at all.

Five patients with hereditary AT deficiency and a prior history of thomboembolism were treated with rhAT on a compassionate use basis for six surgical procedures (Konkle et al, 2003). One patient had two surgical procedures six weeks apart and received rhAT on each occasion. Patients were treated preoperatively, receiving multiple doses of rhAT for 2-16 days. Dosing was determined individually by the investigators with the goal of maintaining an AT activity of 80-150% of normal. AT levels were measured locally using automated chromogenic substrate-based functional assay. Patients were followed for clinical evidence of thrombosis, bleeding, adverse events and development of antibodies to the rhAT.

All six surgical events were successfully treated with rhAT. Dosing was individualized for each patient. In two patients, where initial pre- and post-treatment levels were available, there was a 1.69 and 1.66%/U/kg increase which is similar to the 1.39 and 2.05%/U/kg reported for hpAT. There was no clinical evidence of thrombosis or bleeding and no adverse events related to the drug. Four of the six surgical events were followed up by vascular duplex ultrasound of the lower extremities with no clinical evidence of acute thrombosis (Table 18). Four of the 5 patients, who receive multiple doses of rhAT, were also screened for antibody formation against rhAT several weeks post-operatively. None of the patients developed antibodies to the rhAT.

TABLE 26
Hereditary AT Deficient Patients Treated with RhAT

| | | | Baseline | | Vascular | |
|---|---|---|---|---|---|---|
| | | | AT level | | Duplex | Anti rhAT |
| Subject # | Age | Sex | (%) | Surgery Type | Ultrasound | Antibody |
| 1a | 22 | M | 40 | Laparoscopic | Negative (d5) | |
| | | | | splenectomy | | |
| 1b | 22 | M | 40 | Bilateral hip | | Negative |
| | | | | replacement | | |
| 2 | 47 | F | 49 | Hysterectomy | Negative (d7) | Negative |
| 3 | 36 | F | 58 | C-section | Negative (w6) | Negative |
| 4 | 72 | M | 52 | Coronary Artery Bypass | | |
| 5 | 71 | M | 53 | Knee Replacement | Negative | Negative |

Referring to Table 26, above, it was concluded that the case reports indicated that rhAT can provide effective support for AT-deficient patients who undergo surgery, and is a suitable alternative for hpAT.

Inhibitor Activity

AT is a serine protease inhibitor that inhibits thrombin and Factor Xa, in addition to other coagulation factors (refer to FIG. 2). The addition of heparin increases the inhibitory activity of AT 300 (Factor Xa) to 1,000 (thrombin) fold. The AT binds to a pentasaccharide on the heparin chain that induces an allosterically transmitted conformational change in the reactive center loop of the AT molecule (Meagher et al. 1998). The AT and thrombin then interact to produce a tightly bound TAT complex that is essentially irreversible and cleared quickly from the circulation by a hepatic receptor identified as the LDL Receptor-related Protein (Kounnas et al, 1996). Heparin therefore serves as a catalyst for the AT and thrombin interaction and is not itself involved in inhibition of thrombin.

The specific activity of the rhAT was identical to hpAT (Thrombate) in an in vitro thrombin inhibition assay in the presence of excess heparin (˜6 IU/mg) (Edmunds et al. 1998) and very similar to that reported for Pharmacia's ATnativ (Table 3). The specific activity of the final vialed rhAT product at current manufacturing scale has been consistently measured as ˜7 IU/mg (Table 3) indicating the absence of significant inactive AT.

TABLE 3
Specific Activities of Typical Manufacturing Lots of rhAT

| | Lot 8006 | Lot 8011 | Lot 8015 | Lot 8020 |
|---|---|---|---|---|
| Specific Act. IU/mg | 7.3 | 7.8 | 6.9 | 7.2 |

Recently, two clinical investigators have conducted in vitro studies to assess the behavior of rhAT in assays routinely used to monitor hpAT in patient therapy. Cooper et al (2003) compared rhAT to dilutions of normal pooled plasma in 3 assays: 1) thrombin based assay using 60, 180 and 300 sec incubation of thrombin (Dade Behring kit), 2) factor Xa-based assay (Chromogenix kit), and 3) an hAT ELISA (Dako antibodies). AT level in the rhAT concentrate was assayed against the 8th British Blood Coagulation Factors Standard (plasma) to compare the stated dose with the assayed dose per vial. Heparin binding was also assessed by 2-D electrophoresis with or without heparin in the first dimension and by Heparin-Sepharose gel filtration. The concentration of rhAT in the vial was 89% of the stated value by this thrombin based assay, 85% by Xa-based assay and 119% by the antigen assay. (Gray et al 1999 have established that for most AT standards the functional potency for AT is lower than the antigenic potency and that the most appropriate standard for comparison is the AT concentrate standard and not the AT plasma standard. They also established that there was significant lab variability in the assay results.). Heparin binding showed no observable difference in electrophoretic pattern with or without heparin, but chromatography showed some increased heparin affinity of the rhAT. The investigators concluded that rhAT concentrate can be accurately measured by commercial clinical assays for hpAT using thrombin or factor Xa and that the vial studied contained close to the stated potency with both assays.

Other collaborative studies have been concluded that indicate that that there is high agreement between AT % activity and AT antigen levels (Table 4). The high degree of concordance confirms that rhAT is behaving like endogenous hpAT in these assays since the pre-treatment time point is measuring endogenous hpAT.

TABLE 4
Measurement of AT % Activity and AT Antigen in rhAT Supplementation

| | AT % | |
|---|---|---|
| | activity | AT antigen |
| Pre-treatment with rhAT | 44 | 41 |
| After rhAT | 64 | 64 |
| supplementation | | |
| | 82 | 81 |
| | 200 | 199 |
| | 117 | 115 |

The specific activities of over 15 lots of hpAT from 4 EU manufacturers have been compared (Table 5) by Barrowcliffe et al. (1983) and by Hellstern et. al. (1995). As can be seen by the low specific activities of a few of these products, some AT concentrates had considerable non active AT in their preparations, although they all met the European Pharmacopoiea (1997) definition of >60% native AT in the concentrates. Some of these inactive products have been found to be a cleaved form of AT. It has been suggested that these cleaved AT molecules may be harmful and may cause the release of cytokines (Chang & Harper 1997, Harper et al 1997).

TABLE 5
Specific Activities of EU AT Concentrates

| | | | | Pharmacia | |
|---|---|---|---|---|---|
| (IU/mg) | Alpha | Behring | Immuno | (Old) | Pharmacia (New) |
| Specific Act. | 4.3 (4.2-4.5) | 5.6 (5.2-5.8) | 3.9 (3.4-4.0) | 5.6 (5.4-5.9) | 6.6 (6.2-6.9) |

Numbers in parenthesis are the ranges measured for multiple samples.

Equivalent inhibition for rhAT and Thrombate was seen in an in vitro Factor Xa inhibition assay in the presence of excess heparin (Edmunds et al. 1998). Heparin cofactor activation of rhAT versus hpAT was determined by varying the amount of heparin used in either inhibition assay. RhAT required a lower concentration of heparin than hpAT for inhibition of both enzymes, similar to the β-form of hp AT. Thus, rhAT closely resembles hpAT with respect to its activity for both thrombin and Factor Xa in the presence of saturating levels of heparin (Edmunds et. al. 1998).

Heparin Binding Affinity

Heparin binding to the AT molecule plays a catalytic role in increasing the inhibitory activity of AT toward thrombin and Factor Xa. There are two forms of AT in human plasma having different heparin affinities, but the same inhibitor activity toward thrombin (reviewed in Turk et al 1997; Swendenborg 1998). 85-90% of circulating hpAT has glycosylation on 4 asparagine residues. This fully glycosylated form is referred to as the alpha form. 5-15% of circulating hpAT (referred to as the beta form) lacks glycosylation at Asn 135 and has a 3-10 fold higher heparin affinity than the alpha form (Turk et al 1997). RhAT has a 3-4-fold higher overall heparin affinity than the alpha form of hp AT, due to the glycosylation differences between these molecules. Thus, rhAT has a beta-like heparin affinity. However, in the presence of heparan moieties as found on the surface of vascular endothelial cells or with exogenous heparin supplementation, the alpha, beta and recombinant forms of AT have identical inhibitory activities against thrombin (Turk et al 1997) because the heparin is not itself involved in the inhibition.

By using a Tryptophan fluorescence assay, a four-fold higher affinity for heparin was observed with the rhAT when compared with Thrombate but similar to that reported for the β-form of hp AT. The fluorescence values at saturating heparin were indistinguishable for rhAT and Thrombate (Edmunds et al. 1998).

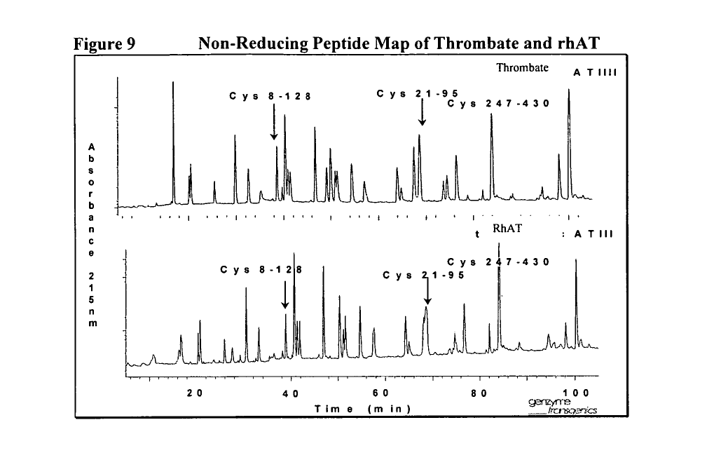

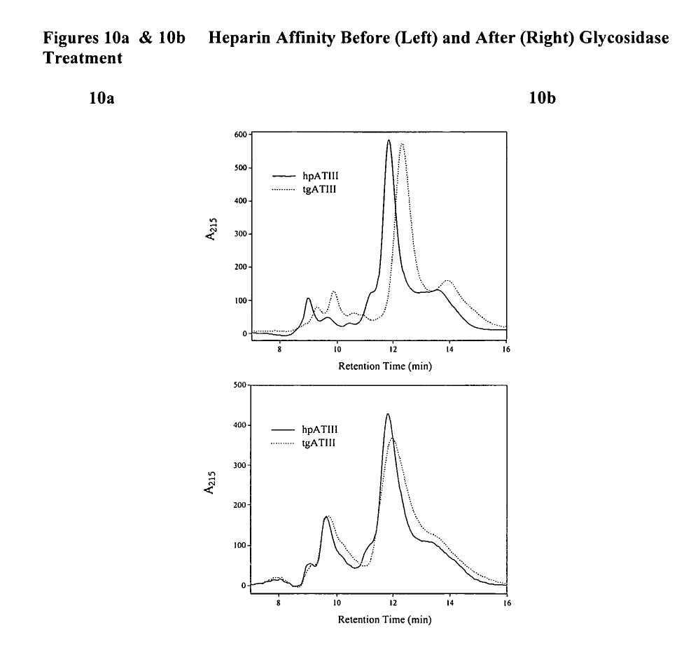

Heparin binds to a glycosaminoglycan on the AT molecule. It has been shown that the presence of carbohydrates on AT particularly at the Asn 135 and Asn 155 sites can greatly affect heparin binding. In carbohydrate remodeling studies (FIG. 10), the heparin binding activity of rhAT (non-heat treated) and hpAT were monitored after incubation with sialidase to remove sialic acid residues and/or endoglycosidase H to remove oligomannose structures (found only on rhAT). Endoglycosidase had little effect on heparin binding affinity of rhAT, however, sialidase treatment caused both hpAT and rhAT to bind heparin more tightly such that they have similar heparin affinities. The increase in heparin affinity was more dramatic for the hpAT since it is more highly sialylated than the somewhat undersialylated rhAT.

Antibody Cross-Reactivity

In vitro, rhAT and hpAT reacted similarly in ELISA (unpublished) and Western Blot assays (Edmunds et al, 1998). Monoclonal and polyclonal antibodies raised to each protein, cross-react with each other and the molecules have proved so far to be immunologically indistinguishable. In contrast, it has been possible to raise specific antibodies to endogenous goat AT (used in the specific goat AT ELISA assay) that do not cross-react with rhAT, although the molecules are >85% homologous.

Immunological Safety

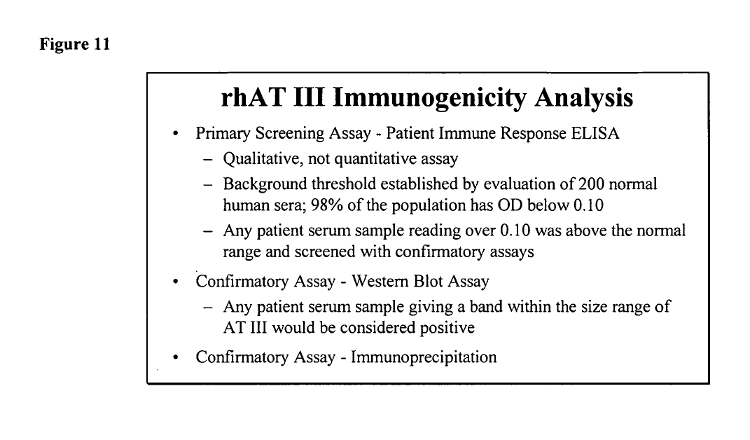

Molecules from one species put into another species, often elicit an immune response to the foreign protein. In pre-clinical animal studies in the rat, dog and monkey, rhAT did elicit an antibody response in these animals. To date, in our human trials over 170 individuals (Table 6) have been treated with rhAT (with 1 or more doses) with no evidence for an immune response as measured by a patient immune response assay (FIG. 11-Table 6) (Richards et al, poster presentation, Innsbruck, Austria, 2003). This indicates that the rhAT is not viewed as foreign by the human immune system and is thus comparable to the individual's own hp AT.

Serum samples from human trial subjects were collected prior to injection of rhAT, as well as, 7 days and 28 days post injection. In the 009 study, samples were collected prior to AT administration and at day 28 and day 60. Patient immune response was evaluated by a plate ELISA with rhAT as the coating agent to detect specific IgG antibodies to rhAT. The color reaction was measured as an optical density at 490 nm using a microplate reader. Any patient serum sample reading over 0.1 was screened with a confirmatory radioimmunoprecipitation (RIP) assay. Two hundred normal human serum samples were used to establish the normal ranges and assay cut-off values.

TABLE 6
Immunogenicity Screen in rhAT III Treated Subjects

| | | No. of | |
|---|---|---|---|
| | | Patients | No. with |
| Study | Sampling Days | Treated | Antibodies |
| 9601 Phase I | Pre-administration & day 28 | 15 | 0 |
| 0801 Phase II | Pre-administration & ≧day 21 | 30 | 0 |
| 903 Comparability | Pre-administration & day 28 | 33 | 0 |
| 502 & 504 Phase III | Pre-administration & day 28 | 55 | 0 |
| 006 Crossover (2 | Day before administration & | 23 | 0 |
| doses) | day 28 (for both doses | | |
| Totals | | 156 | 0 |

Control subjects and those that received hpATIII in the 903 trial are not included in this table.

Viral Safety

By virtue of its source and manufacturing methods, rhAT is inherently unlikely to transmit human blood-borne viruses and other plasma derived human infectious agents. Moreover, no human-derived protein is added during the production, isolation or formulation of rhAT. This is in contrast to two EU approved hpAT products, that contain added human serum albumin in their final formulation.

The first step in the viral safety strategy for hpAT is donor selection. This is accomplished at blood collection centers through a questionnaire to ascertain whether the donor poses a risk to the blood supply and through repeat donor historical information. Since GTC's goatherd is closed and highly controlled, a high level of donor control and viral & non-viral disease testing is a key parameter in GTC's viral safety strategy for rhAT.

The plasma pools used by all manufacturers of hpAT are screened for a limited number of specific human viruses or viral exposure (e.g. HIV, HCV, Hepatitis). In addition to the high level of donor control and goat testing, the milk containing the rhAT is screened in vitro on three or four cell cultures (e.g. human MRC-5, monkey Vero, BHK-21 and goat turbinate) for evidence of adventitious viruses that cause cytopathic effects or hemadsorption or hemagglutination with various red blood cells. This assay would detect a broad range of viruses, including any emerging unknown virus, that may be present in the milk pool used as the starting material for production of rhAT. Additionally, immunofluorescne assays for specific viruses of concern (e.g. West Nile Virus) have been added to the viral screening of the milk pools. To date all milk pools from the GTC Farm (including rhAT) have tested negative in the in vitro cell line screening assay.

The manufacturing processes for all the hpAT differ in their details depending on the manufacturer. Some begin with Cohn Fraction IV-1 and others with Fraction II+III supernatant. Each of the processes also includes at least one viral inactivation step (heat treatment) (Table 7). Pharmacia's ATnativ includes two inactivation steps. The rhAT process includes a terminal, validated viral inactivation step (dry heat −80° C. for 72 hr) that is placed at the end of the process after lyophilization.

TABLE 7
Viral Inactivation Methods for AT Concentrates

| Product | Viral Inactivation Method* |
|---|---|
| Immuno - Antithrombin III Immuno | Liquid pasteurization |
| | (60° C. for 10 hr) |
| Pharmacia - Atenativ | Liquid pasteurization |
| | (60° C. for 10 hr) + |
| | solvent/detergent |
| Aventis Behring - Kybernin | Liquid pasteurization |
| | (60° C. for 10 hr) |
| Grifols - Anbin | Liquid pasteurization |
| | (60° C. for 10 hr) |
| Bayer - Thrombate | Liquid pasteurization |
| | (60° C. for 10 hr) |
| GTC Biotherapeutics - rh AT | Dry heat (80° C. for 72 hr) |

*Information contained in package insert for each product.

At least one hpAT manufacturer has recently incorporated a nanofiltration step for viral removal in their process. A small human pharmacokinetic clinical trial with this material has demonstrated no change in clinical parameters for this product after inclusion of the nanofiltration step (Marzo et al, 2002). GTC has developed a nanofiltration step for viral removal for inclusion in the current rhAT process between the heparin affinity column and the ion exchange column. While it is unlikely that nanofiltration will alter the rhAT in any way, the placement of this step in the purification process took into consideration that the two columns that follow the nanofiltration step would most likely remove any altered material. Appropriate biochemical comparability testing and bioequivalence testing, including rat pharmokinetic (“PK”) and BD study & a human PK have been performed to ensure that the final rhAT is comparable to that used in the rhAT clinical trials to date. The validated viral removal capacity of the current rhAT manufacturing process is shown in Table 8 below.

TABLE 8
Validation of Viral Removal or Inactivation by the RhAT Process

| | | Xenotropic | | | | |
|---|---|---|---|---|---|---|
| | Pseudorabies | Murine | Human | Porcine | Polio | Mouse |
| Process Step | Virus | Retrovirus | Adenovirus | Parvovirus | Virus* | Adenovirus* |
| 500-kD TFF | ≧5.1 | ≧3.7 | ≧5.3 | 2.4 | 4.1 | 3.5 |
| Heparin Hyper-D | 1.8 | 1.2 | <1.0 | 1.4 | 4.0 | 2.3 |
| Pall DV-20 Filter | ≧4.8 | ≧3.8 | ≧6.3 | ≧3.7 | ND | ND |
| ANX-Sepharose | 3.6 | 1.0 | ≧7.1 | 1.1 | 2.4 | <1.0 |
| Methyl HyperD | ≧5.6 | ≧3.5 | ≧4.8 | ≧5.7 | ≧5.2 | ≧2.7 |
| Heat Treatment | 2.8 | ≧5.0 | ≧1.8 | 2.4 | ≧1.9 | ND |
| Total Reduction | ≧23.7 | ≧18.2 | ≧25.3 | ≧16.7 | ≧17.6 | ≧8.5 |

*Polio virus and mouse Adenovirus were only done once in preliminary validation runs. All the others were run in duplicate and in some cases three times.
ND = Not Determined

The results of the viral validation studies demonstrate that a significant virus reduction of ≧8.5 to ≧25.3 log₁₀ was accomplished across the distinctly different modes of the rhAT process. These data strongly support the conclusion that rhAT produced using these specific steps is safe for human use with respect to potential adventitious agent contamination and is similar to data for hpAT concentrates except that no human infectious viruses are present in the source material for rhAT as determined by in vitro cell line screening.

Published information on validation for viral removal or inactivation for each of the commercial hpAT's is not readily available, although isolated data for a few viruses can be found (Table 9).

TABLE 9
Validation of Viral Removal or Inactivation for AT Concentrates

| | | | Adenovirus | | |
|---|---|---|---|---|---|
| AT | Retrovirus | Herpes | (Non | | |
| Concentrate | (Enveloped) | (Enveloped) | Enveloped) | Parvovirus | Others |
| ATnativ | ≧13.5 (HIV-1) | ≧11.7 (IBR) | ≧7.1 (HAV) | | ≧11.4 (Sindbis) |
| Anbin | ≧11.7 (HIV-1) | ≧8.1 (BHV) | ≧6.0 (EMC) | | ≧7.9 (BVDV) |
| Antithrombin | ≧13.3 (HIV-1) | ≧14.6 (PRV) | ≧5.4 (HAV) | | ≧10.3 (FSMEV) |
| III Immuno | | | | | ≧13.0 (ERV-1) |
| Kybernin | ≧6.7 (HIV-1)* | ≧6.8 (HSV)* | | 10.4 (B19) | ≧4.5 (CMV)* ≧6.9 |
| | | | | | (polio)*, 4.0 |
| | | | | | (vaccinia)* |
| RhAT | ≧18.2 (XMR) | ≧23.7 (PRV) | ≧25.3 (HAV) | ≧16.7 (PPV) | ≧17.6 (Polio) |
| | | | ≧8.5 (MAV) | | |

HAV—human adenovirus,
BHV—bovine herpes virus,
BVDV—bovine diarrhea virus,
EMC—Encephalomycarditis virus,
PRV—pseudorabies virurs,
B19—human parvovirus,
CMV—cytomegalavirus,
HSV—Herpes Simplex Virus,
XMR—xenotrophic murine retrovirus,
MAV—mouse adenovirus,
PPV—porcine parvovirus.
*Pasteurization step only - results.

Prion Safety

Transmissible spongiform encephalopathies (TSE), such as nvCJD in humans, BSE in cattle and scrapie in sheep and goats, also must be considered in assuring the safety of products made from human or ruminant sources. Human donors are monitored for CJD and nvCJD and potentially contaminated blood, plasma pools and products made from them have been recalled or traced when a contributing donor has been diagnosed with CJD. All GTC goats are certified free of scrapie in the 5 yr USDA Voluntary Scrapie Certification Program and various risk minimization measures have been instituted to reduce any potential risk from this TSE in this highly controlled closed goat population. In addition, the rhAT purification process has been validated for its ability to remove a minimum of ≧11.3 log₁₀ scrapie (Table 10). While not included in the validation studies the, the Pall DV-20 filter has been reported to remove ≧2.8 log₁₀ prions (Aranha and Larson 2002), which provides an additional presumptive level of safety.

TABLE 10
Scrapie Reduction by the rhAT Purification Process

| Unit Operation | |
|---|---|
| Reduction | Log10 |
| 500-KDTFF | 2.0 |
| Heparin HyperD | 2.2 |
| ANX-Sepharose | ≧3.3 |
| Methyl HyperD | ≧3.8 |
| Cumulative Reduction | ≧11.3 |

These data strongly support the conclusion that rhAT purification process is capable of removing scrapie. To the best of our knowledge, similar studies have not been performed on the actual plasma AT purification processes even though the risk of CJD transmission is greater than that for scrapie.

Prion Clearance Capability of the rhAT

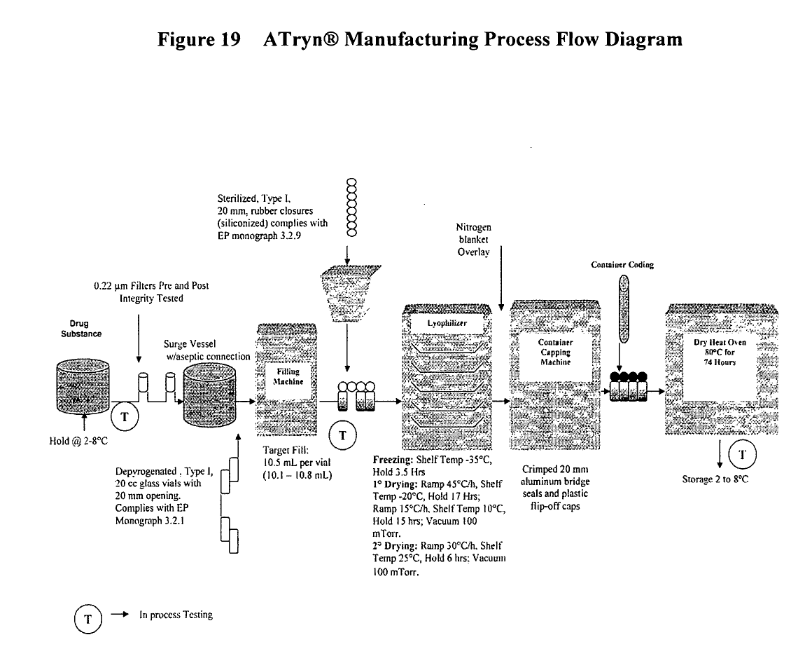

Once the milk containing hrAT is collected from the goats, it must be processed to isolate the rhAT from the milk protein contaminants. FIG. 19 shows the rhAT manufacturing process. A scrapie removal evaluation study was conducted using Good Laboratory Practices at Primedica Laboratories (Rockville, Md.) to evaluate the prion clearance capability of the process steps of the invention as evaluated for viral removal/inactivation. The terminal dry heat treatment step was not included since it has no reported prion inactivation capability.

Prior to study commencement, control experiments examined the effects of scale down and negative control spikes (normal brain homogenate extract.) on the behavior of rhAT through the process steps. The control experiments also included assessment of the effects of the selected process buffers on the bioassay used for scrapie quantitation.

The ME7 strain of mouse adapted scrapie was used in spike and process step recovery experiments. Clarified scrapie mouse brain homogenates were prepared for spiking. Process samples were spiked with homogenate and each process step run to produce test article. C57BI/6 mice were intracerebrally inoculated with the test articles and monitored for clinical or behavioral changes. At 9 to 10 months, initial mortality results were obtained and at 14 to 16 months, surviving animals were killed and their brains histopathologically analyzed for signs of scrapie lesions. The cumulative scrapie removal capacity of the purification process is >11.3 log₁₀ reduction and the step reductions are shown in Table 11.

TABLE 11
Scrapie Reduction by the rhAT Purification Process

| Unit Operation | Log₁₀ Reduction |
|---|---|
| Tangential Flow Filtration | 2.0 |
| Affinity Chromatography | 2.2 |
| Anion Exchange Chromatography | ≧3.3 |
| Hydrophobic Interaction Chromatography | ≧3.8 |
| Cumulative Reduction | ≧11.3 |

Although the Pall DV-20 viral filter has not been validated for scrapie removal in the rhAT process, this filter has been reported to remove ≧2.8 logio of prions (Aranha and Larson 2002), which provides an additional presumptive level of safety. Therefore, the ≧11.3 log¹⁰ reduction is the minimal reduction factor for the current process. If one takes into account the reported reduction by the viral filter, then ≧14.1 log¹⁰ is the minimal reduction factor for the current process.

Quantitative TSE Risk Assessments for rhAT

In 1994, the German Federal Ministry of Health issued guidelines for the German Pharmaceutical Industry for a TSE risk assessment for all medicinal products made from animal sources or utilizing animal derived products in their manufacturing process (Federal Bulletin No 40, 26 Feb. 1994). These guidelines were updated in 1995. They included a quantitative risk assessment to be applied to all such products before their consideration for registration. A score of ˜20 was required for product registration in Germany. RhAT made from milk obtained from transgenic goats and using the process of the invention has been evaluated in this assessment and the numerical safety factor has been calculated for each rhAT clinical indication. The primary risk of TSE contamination in final dosage rhAT comes from the rhAT drug substance itself. None of the components represent a significant risk of TSE. It is also important to remember that unlike BSE, the TSE of goats is scrapie, which has never been shown to be transmitted to humans, even though the disease has been known for over 250 years.

Heparin Resistance Indication

RhAT was first used in clinical trials for the treatment of heparin resistance in patients undergoing cardiopulmonary bypass grafting. The effective human dosage determined from the clinical trials was 75 IU/kg. RhAT has an average specific activity of 7 IU/mg and therefore the human dose was approximately 10 mg/kg. For a 70-90 kg patient that translated into 0.7-0.9 kg of rhAT administered on a single occasion. The purification yield for the rhAT process is approximately 50% and the product is present in the milk at an average concentration of 2 gm per liter. Therefore, a single patient daily dose is made from approximately 1 Liter of goat milk. The purification process has been validated to remove ≧11 log¹⁰ of scrapie (Genzyme Study No. TR-PPR-903). With this information, the risk factor for the heparin resistance indication can be calculated. RhAT's cumulative safety score is 30 (32 if published filter data included with proper I log reduction since not actually verified in study) (Table 12), which far exceeds the safety score of ≧20 that was required for registration in Germany.

TABLE 12
BfArM Risk Assessment for Spongiform Encephalopathies (Heparin
Resistance Indication)

| Parameter | Comment | Safety Factor |
|---|---|---|
| Origin of animals | Closed Certified scrapie free | 8 |
| | goats from NZ & US, fed no | |
| | non-exempt feedstuff | |
| Starting material | Milk | 8 |
| Inactivation/removal | Process validation using | 11 (13) |
| | mouse bioassay | |
| Start material per daily | <1 kg to >100 g | 0 |
| allowance | | |
| Number of daily allowances | Occasional 1-9 daily doses/yr | 2 |
| Route of administration | Intravenous | I |
| Cumulative safety score | >20 is required | 30 (32) |

(numbers in parenthesis are safety factor adding in published data on Pall DV-20 viral removal filter after discounting the removal by 1 log as spec˜fled in the guidance document.)

Product Considerations

All commercial hpATs are available as sterile, stable, lyophilized preparations. Of the hpATs, two (Immuno and Pharmacia) add human serum albumin to the preparations as an excipient. Like rhAT, the remaining products do not contain added albumin. All preparations indicate recommended storage at 2-8° C., which is also the recommended storage temperature for rhAT. All commercial AT concentrates have a 2-3 year shelf life that agrees with the proposed shelf life for rhAT.

In Vitro and In Vivo Studies Comparing the Biological Activity of rhAT and hpAT

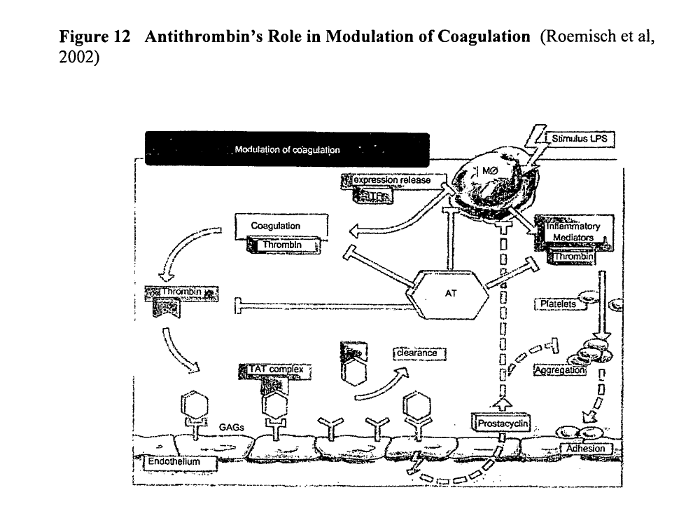

AT is a complex protein with multiple biologically important activities. It is the most critical modulator of coagulation (FIG. 12) and has potent anti-inflammatory properties (FIG. 13) independent of its effects on coagulation (recently reviewed in Roemisch et al, 2002). Various collaborators have used rhAT in in vitro and in vivo animal studies, some of which used hpAT as a direct comparator. Some of these studies have been published and others are documented as personal communications with GTC Biotherapeutics scientific staff. The coagulation modulatory studies will be described first and then the anti-inflammatory studies in the sepsis models.

In Vitro ACT Prolongation

In vitro data supports consistent biological activity of hpAT and rhAT. Heparin requires AT to be effective in anticoagulation. However, patients on continuous small-dose heparin pre-operatively have decreased levels of AT. These patients may be heparin resistant and require supplementation with AT to restore their heparin responsiveness. Levy et al (2000) evaluated and established the rationale for restoration of anticoagulation responses in the clinical setting. Blood samples were obtained from cardiac surgical patients including 22 patients receiving heparin and 21 patients not receiving heparin preoperatively. AT activity was 69% in patients receiving heparin and 92% in patients not receiving heparin. Heparin was added to the blood in increasing concentrations and kaolin-activated clotting times (ACTs) were determined with and without supplementation with 0.2 U/mL hpAT (Thrombate III, Bayer, Inc., Elkhart, Ind.) to mimic fresh frozen plasma administration. In response, ACT, the standard measurement for monitoring anticoagulation, were significantly prolonged with hpAT supplementation while additional heparin alone failed to produce any further increases in ACT values.

Subsequently, Levy et al also evaluated the effect of rhAT on in vitro coagulation in blood from cardiac surgical patients in the same laboratory. This study was conducted by Dr. J. H. Levy at Emory University School of Medicine, Division of Cardiothoracic Anesthesiology and Critical Care, The Emory Clinic, Atlanta, Ga. Forty-two (42) adult patients electively scheduled for cardiac surgery requiring cardiopulmonary bypass were studied. Blood samples were obtained following vascular access from patients receiving intravenous heparin for at least 48 hours pre-operatively and from control patients not receiving heparin. Activated clotting times (ACT) were measured to determine the adequacy of heparin responses.

To determine the concentration of rhAT that produces a maximal increase in ACT, 0.4 mL of blood was placed in ACT cartridges to determine baseline ACT, and into cartridges that contained heparin equivalent to a 300 U/kg dose (4.1 U/mL). Similar amounts of blood were also added to cartridges that contained the same amount of heparin plus increasing concentrations of added rhAT, of 0.1, 0.2, 0.5 and 1.0 unit/mL. The ACT values were recorded. Baseline AT activity levels were also analyzed in both groups. The mean AT activity in the patients on heparin was 75% (range 19.5-97.6%) and the mean AT activity in the patients not receiving heparin was 95% (range 80-111%).

Recombinant human AT produced a significant increase in ACT in both groups of patients. The mean change in ACT for each one of the AT concentrations used is shown in Table 13.

TABLE 13
Summary of Mean ACT Values for Each Concentration of RhAT

| | | Heparin | | | | |
|---|---|---|---|---|---|---|
| Patients | Baseline | 3 mg/kg | rhAT (0.1) | rhAT (0.2) | rhAT (0.5) | rhAT (1.0) |
| NOT on Heparin | 126.775 | 524.575 | 647.225 | 704.45 | 720.05 | 790.425 |
| ON Heparin | 147.2 | 444.075 | 600.975 | 631 | 635.35 | 711.075 |

In conclusion, the data demonstrated that in blood from cardiac surgical patients addition of rhAT increased the heparin-dose response (measured as increased ACT) similar to previously reported results with Thrombate (above).

RhAT and hpAT in Animal Studies

A variety of pre-clinical animal studies have been performed utilizing rhAT including single and repeat dose PK and toxicity testing in rats, dogs and monkeys. However, only a limited number of these studies used an hpAT comparator. These studies will be briefly summarized below.

Rat PK Study

The objective of this study was to evaluate the clearance of three doses of rhAT and a single dose of human plasma AT in the Sprague Dawley rat. As an addendum to this study, 2 additional doses of hpAT were studied to complete the dose response comparison.

The clearance of three doses (12.5, 41.7 and 125 mg/Kg) of recombinant human AT and three doses (20, 42 and 125 mg/kg) of human plasma AT (Thrombate) from rat serum was examined. Model independent analysis of the pharmacokinetic data indicated that clearance of rhAT from rat serum was affected by dose. Low doses of rhAT had a shorter mean residence time (MRT) and a greater clearance rate (CL) than high doses of rhAT (Table 14). RhAT (even at 125 mg/kg) was cleared more rapidly from rat serum than hpAT.

TABLE 14
Model Independent Parameters Defining AT Clearance In Rats

| | Dose | Animals | MRT | CL (mL/ | Vss |
|---|---|---|---|---|---|
| AT | (mg/Kg) | (No.) | (minutes) | min/Kg) | (mL/Kg) |
| rh | 13 | 2 | 24 | 2.6 | 76 |
| rh | 42 | 2 | 36 | 1.2 | 46 |
| rh | 125 | 2 | 79 | 0.5 | 42 |
| human plasma | 20 | 4 | 146 | 0.5 | 71 |
| human plasma | 42* | 3 | 153 | 0.3 | 45 |
| human plasma | 125* | 3 | 220 | 0.2 | 48 |

*Experiments that measured the pharmacokinetics of these doses of human plasma AT were performed as an addendum to the original study.

A single equation was developed that fit the pharmacokinetic data for rhAT, regardless of dose. The model invoked 3 clearance mechanisms: first order clearance by kidneys; receptor mediated compartmentalization by heparin-like proteoglycans; and receptor mediated clearance by the asialo-receptor. The pharmacokinetic data for hpAT was fit by changing the constants in the pharmacokinetic equation describing rhAT clearance to reflect predicted effects of sialylation on first order clearance by kidneys, predicted effects of sialylation on clearance by asialo receptors, and the lower affinity of the hpAT molecule for heparin.

Monkey PK Study

The objective of this study was to compare the pharmacokinetics of radiolabelled rhAT with radiolabelled hpAT (Thrombate) in cynomolgus monkeys.

There were two groups of 5 male cynomolgus monkeys each. Group 1 animals were administered a bolus injection of ¹²⁵I-hpAT at a dose of ˜3 mg hpAT/kg and a dose volume of 1 mL/kg. Group 2 animals were administered a bolus injection of ¹²⁵I-rhAT at a dose of 3 mg rhAT/kg and a dose volume of 1 mL/kg. Blood sampling for pharmacokinetic analysis were obtained at various times post injection, and sera were analyzed for total cpm and TCA precipitable cpm. The data (Table 15) indicate that ¹²⁵I-rhAT is cleared much more rapidly from sera in cynomolgus monkeys than ¹²⁵I-hpAT when administered in trace amounts with 3 mg/kg non-radiolabeled carrier protein. There is a 4-fold difference in the mean residence time (MRT) and a 6-fold difference in clearance (CL) rates between these molecules. It is possible that these difference will become smaller at higher doses of AT. The 3 mg/kg dose of carrier protein is equivalent to 0.5 U/kg of exogenous AT.

TABLE 15
Model Independent Analysis of Pharmacokinetic Data from Cynomolgus Monkeys.

| | | | AUC* | AUMC* | | | |
|---|---|---|---|---|---|---|---|
| Test | | Dose* | (cpm- | (cpm-hr- | | CL | Vdss |
| Article | Animal # | (mg/kg) | hr/ml) | hr/ml) | MRT (hrs) | (ml/hr/kg) | (ml/kg) |
| hpAT | #1001 | 3 | 2.0 × 10⁷ | 5.3 × 10⁸ | 26.3 | 2 | 52.2 |
| | #1002 | 3 | 2.3 × 10⁷ | 7.0 × 10⁸ | 29.9 | 1.7 | 51.3 |
| | #1003 | 3 | 2. × 10⁷ | 6.9 × 10⁸ | 30.3 | 1.8 | 53.5 |
| | #1004 | 3 | 2.4 × 10⁷ | 7.9 × 10⁸ | 32.4 | 1.6 | 53.4 |
| | #1005 | 3 | 1.7 × 10⁷ | 5.0 × 10⁸ | 29 | 2.3 | 67.6 |
| | MEAN | 3 | 2.1 × 10⁷ | 6.4 × 10⁸ | 29.6 | 1.9 | 55.6 |
| | SD | | 2.9 × 10⁶ | 1.2 × 10⁸ | 2.2 | 0.3 | 6.8 |
| rhAT | #2001 | 3 | 3.2 × 10⁶ | 1.9 × 10⁷ | 5.8 | 12.4 | 72.1 |
| | #2002 | 3 | 2.2 × 10⁶ | 1.1 × 10⁷ | 4.9 | 18.2 | 89.2 |
| | #2003 | 3 | 4.0 × 10⁶ | 4.6 × 10⁷ | 11.6 | 10.1 | 116.9 |
| | #2004 | 3 | 3.1 × 10⁶ | 1.9 × 10⁷ | 6.1 | 13 | 78.8 |
| | #2005 | 3 | 2.8 × 10⁶ | 1.7 × 10⁷ | 6.1 | 14.2 | 86.6 |
| | MEAN | 3 | 3.1 × 10⁶ | 2.2 × 10⁷ | 6.9 | 13.6 | 88.7 |
| | SD | | 6.5 × 10⁵ | 1.4 × 10⁷ | 2.7 | 3.0 | 17.1 |

*AUC calculated between 0-168 hr for hpAT and between 0-48 hr for rhAT.
*AUMC calculated between 0-168 hr for hpAT and between 0-48 hr for rhAT.

A Safety Study of the Interaction of Heparin and rhAT in Sprague Dawley Rats

The objective of this study was to evaluate the safety and pharmacology of rhAT infusion followed by a bolus heparin administration in rats. A comparative study was also performed for evaluation of equivalency between rhAT and hp AT. Table 16 is a summary of the experimental design.

TABLE 16
Summary of Study Experimental Design

| | Number of | | Treatment Administration | | | | |
|---|---|---|---|---|---|---|---|
| Group | Animals | | | Dose | | Dosing | Observation |
| No. # | Males | Females | Substance | Level | Route | Regimen | Period |
| 1 | 15 | 15 | Glycine/Citrate | 0 mg/kg | IV | 30 minutes at | Up to Day 8 |
| | | | buffer | | infusion | 10 mL/kg | |
| | | | Sodium | 300 U/kg | IV bolus | Once | |
| | | | Heparin | | injection | | |
| 2 | 15 | 15 | rhAT | 36 mg/kg | IV | 30 minutes at | |
| | | | | | infusion | 10 mL/kg | |
| | | | Sodium | 300 U/kg | IV bolus | Once | |
| | | | Heparin | | injection | | |
| 3 | 15 | 15 | rhAT | 210 mg/kg | IV | 30 minutes at | |
| | | | | | infusion | 10 mL/kg | |
| | | | Sodium | 300 U/kg | IV bolus | Once | |
| | | | Heparin | | injection | | |
| 4 | 15 | 15 | rhAT | 360 mg/kg | IV | 30 minutes at | |
| | | | | | infusion | 10 mL/kg | |
| | | | Sodium | 300 U/kg | IV bolus | Once | |
| | | | Heparin | | injection | | |
| 5 | 15 | 15 | Glycine/Citrate | 0 mg/kg | IV | 30 minutes at | |
| | | | buffer | | infusion | 10 mL/kg | |
| | | | Sodium | 300 U/kg | IV bolus | Once | |
| | | | Heparin | | injection | | |
| 6 | 15 | 15 | hpAT | 36 mg/kg | IV | 30 minutes at | |
| | | | | | infusion | 10 mL/kg | |
| | | | Sodium | 300 U/kg | IV bolus | Once | |
| | | | Heparin | | injection | | |
| 7 | 15 | 15 | HpAT | 210 mg/kg | IV | 30 minutes at | |
| | | | | | infusion | 10 mL/kg | |
| | | | Sodium | 300 U/kg | IV bolus | Once | |
| | | | Heparin | | injection | | |
| 8 | 15 | 15 | hpAT | 360 mg/kg | IV | 30 minutes at | |
| | | | | | infusion | 10 mL/kg | |
| | | | Sodium | 300 U/kg | IV bolus | Once | |
| | | | Heparin | | injection | | |

IV = intravenous

The study consisted of eight groups of Sprague-Dawley rats (30 rats/group, 15 male and 15 female). All groups were administered a thirty minute intravenous infusion at a constant volume of 10 mL/kg of vehicle (glycine citrate buffer, Groups 1 and 5), rhAT (36 mg/kg (Group 2), 210 mg/kg (Group 3) or 360 mg/kg (Group 4)) or hpAT (36 mg/kg (Group 6), 210 mg/kg (Group 7) or 360 mg/kg (Group 8)). Immediately following the infusion, all groups received a single IV bolus injection of sodium heparin at 300 U/kg. Groups 6 through 8 using the hpAT were added after studies with rhAT had been initiated. Therefore, a separate vehicle control group (Group 5) was included. Animals administered the hpAT infusion were not size- and age-matched to the rhAT rats. Animals were monitored for change in body weight and any signs of adverse clinical events for up to seven days following treatment.

Clinical observations associated with rhAT were transient facial and limb swelling in Group 3 and 4 rats infused with 210 mg/kg or 360 mg/kg. Swelling in the intermediate and high dose rhAT groups was observed at approximately 1 hour post-dose and resolved by Day 2. This observation was not evident in hpAT dose groups. There were no biologically relevant effects on serum chemistry parameters in animals administered rhAT or hpAT when compared to the concurrent control group. No physiologically relevant changes in hematology were observed in any of the treatment groups. Body weights in all treatment groups were slightly lower at 4 Days when compared to pretreatment values. Since this was evident in both control and hp or rhAT groups, these data suggests this was not treatment related. By Day 8, the body weight all male groups exceeded pretreatment values. In contrast, only mid dose hpAT females had exceeded their pre-treatment body weights. Any statistical differences noted between rhAT and hpAT treated animals were attributed to the 1.5 week difference in age between Groups 1 through 4 and Groups 5 through 8.

No differences in platelet aggregation to ADP were observed at any of the time points evaluated in either male or female rats in any treatment group. Thrombin times were consistently elevated in a comparable manner between rhAT and hpAT dosed animals at 10 and 60 minutes post-dose. Prothrombin time (PT) increased in a dose-dependent manner for both rhAT and hpAT 10 minutes post-dose. Group 4 (360 mg/kg rhAT) males and females exhibited the greatest increase in PT (+82% and +109%) at 10 minutes compared to control treatment (17.1 vs. 31.1 seconds for males Groups 1 and 4, respectively; 17.1 vs 37.9 seconds for female Groups 1 and 4, respectively). A similar finding was observed at 10 minutes in animals dosed with hpAT. A statistically significant prolongation in PT time was still evident at 60 minutes. All PT times measured later than 1 hour in all treatment groups were considered normal.

Activated partial thromboplastin (APTT) times were uniformly elevated to greater than 212 seconds in all rhAT and hpAT treatment groups at 10 minutes post-dose. APTT values were comparable to controls by 24 hours. No consistent pharmacologic effect on ACT values was evident in any of the treatment groups.

No gross necropsy observations were noted at Day 4 or 8 of study. Thus, infusion of rhAT or hpAT followed by bolus IV injection of heparin was well tolerated up to a dose of 360 mg/kg. The expected pharmacologic effects of prolonged PT and APTT was evident immediately post dose in both rhAT and hpAT animals.

Anti-Inflammatory Properties of rhAT and hpAT

Since there are no commercially available animal models for hereditary AT deficiency, the only way to ascertain the biological consistency of the rhAT compared to hpAT in animal disease models is to utilize in vitro and in vivo models for acquired AT deficiency, such as E coli induced sepsis. Although in these models the exact contributions of the anti-coagulant and anti-inflammatory properties of AT are still somewhat controversial (reviewed in Risberg 1998), they do allow for some biological comparison of the properties of rhAT with hpAT.

AT's role in modulation of the sepsis cascade has been elucidated by various animal and in vitro studies. Table 17 summarizes some of the salient areas where AT has been shown to have an impact in various sepsis models. In some cases, hpAT and rhAT were studied head to head in these studies, while in other cases, the data comes from separate studies. So far, hpAT and rhAT seem to exert similar impacts on these parameters associated with bacterial or endotoxin induced sepsis.

TABLE 17
Effects of hpAT and rhAT on Various Parameters Associated with Sepsis
in Model Systems.

| Parameter | Sepsis Control | HpAT | RhAT |
|---|---|---|---|
| Mortality | 80-100% | Promotes | Promotes |
| | (1, 2, 10) | survival | survival (2) |
| | | (1, 10) | |
| AT Levels | ↓↓ (1, 2) | | |
| E. coli clearance | | ↑ (1) | ↑↑ (2) |
| Fibrinogen levels | ↓ (1, 2, 10, 14) | ↑ (1, 10) | ↑ (2, 14) |
| TAT levels | ↑ (2, 7) | | ↑↑ (2) |
| TPA levels | ↑↑ (2) | | ↓↓ rise (2) |
| PAP levels | ↑↑ (2) | | ↓↓ rise (2) |
| PAI-1 levels | ↑ (2, 7) | | ↑ (2, 7) |
| TNF levels | ↑ (2) | | ↑↑ (2) |
| IL-10 levels | ↑↑ (2) | | ↓↓ rise (2) |
| IL-6 levels | ↑↑ (2, 3, 5, 6) | ↓↓ rise (3, 5, | ↓↓ rise (2, |
| | | 6) | 6) |
| IL-8 levels | ↑↑ (2, 6) | ↑ (6) | ↓↓ rise (2, |
| | | | 6) |
| Tissue Factor levels | ↑ (3, 5, 6) | ↓↓ rise (3, 5, | ↑ (6) |
| | | 6) | |
| Prostacyclin Release | | ↑ (8, 9, 12) | ↑ (7, 12) |
| Platelet Aggregation | ↑ (12) | | ↑ (12) |
| Neutrophil Adherence | ↑ (13) | | ↓ rise (13) |
| Neutrophil Chemotaxis | ↑ (4, 11) | ↓ (4, 11) | ↓ (11) |
| Leukocyte Rolling | ↑ (7, 8) | ↓ rise (8) | ↓ rise (7) |
| Leukocyte Adhesion | ↑ (7, 8, 9) | ↓ rise (8, 9) | ↓ rise (7) |

Numbers in parenthesis refer to references. See appendix for reference key.
↑ indicates that parameter is increased;
↓ indicates that parameter is decreased or baseline,
↓ rise indicates that the AT inhibits the rise due to sepsis.
Multiple arrows indicate increased effectiveness.

Human plasma AT has been shown to prevent the lethal effects of experimentally induced sepsis in several animal models (Taylor et al, 1988, Dickneite & Paques, 1993, and Kessler et al, 1997) and to block cytokine production in vitro. To date, there have been 4 dose response studies performed with rhAT in rats (unpublished) and baboons (Minnema et al., 2000) that have been lethally challenged with E. coli, K. pneumoniae or lipopolysaccharide (LPS). Three studies compared the effects of hpAT with rhAT in a rat sepsis model at several dosages of either hpAT or rhAT. Sepsis was induced by LPS or K. pneumoniae both preparations were equally effective in preventing sepsis and septic shock in these models. Additional studies have been performed demonstrating protective effects of rhAT in a smoke inhalation sepsis model in sheep and in a xenotransplantation model in primates where vascular injury may be accompanied by a form of consumptive coagulopathy in recipients.

Manufacture

TABLE 18
ATryn ® Batch Formula

| Component | Quality Standard | Description | Quantity | |
|---|---|---|---|---|
| Antithrombin alfa | Non-compendia | Active Ingredient | 300 | g |
| Containers | EP | Type 1 Glass, 20 mL, | 1250 | |
| | | 20 mm | | |
| Nitrogen | EP | Overlay | QS | |
| Rubber Closures | EP | 20 mm, Type 1 rubber | 1250 | |
| Aluminum Seal | Non-compendia | 20 mm, plastic flip cap | 1250 | |

The theoretical number of containers produced from this representative 300 gm batch size is approximately 1250 containers whereas the actual number is less than that due to production line setup requirements, mechanical losses in filling lines and collection of samples for in-process and finished product testing.

Description of Manufacturing Process and Process Controls

The manufacturing process used to produce ATryn® is a traditional aseptic fill, lyophilization and finish operation. Key steps in the manufacture of the dosage form are sterile filtration of bulk formulated solution, aseptic filling of the containers to target fill volume, lyophilization to achieve a suitable cake having low moisture and heat treatment for viral inactivation. An example of the type of equipment used and the working capacity, where relevant are provided in Table 19.

TABLE 19
Example Manufacturing Equipment and Working Capacity

| | | Approximate Working |
|---|---|---|
| Equipment | Size | Capacity |
| Steam Sterilizer | 0.8 M³ | Material dependent |
| Dry Heat Oven | 1.5 M³ | 6490 vials |
| High Speed Filler | N/A | 60 vials/minute |
| Edwards Lyophilizer | 5 M² | 4750 vials |
| Container Capping Machine | N/A | 150 vials/minute |
| Container Coding Machine | N/A | 150 vials/minute |
| Label Machine | N/A | 150 vials/minute |
| Product Bulk Tank | 80 L | 60 L |
| Surge Vessel | 15 L | 7 Kg |
| Heat Treatment Oven | 1.5 M² | 5508 vials |

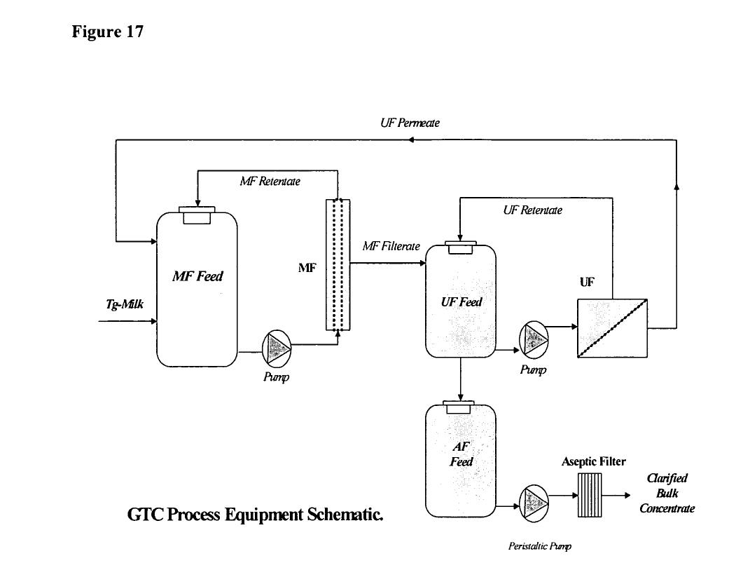

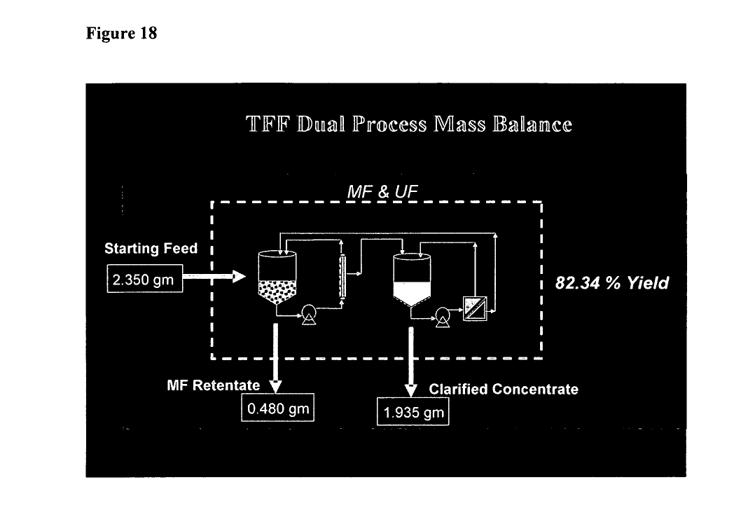

A target volume of approximately 10.5 mL of the bulk formulated antithrombin alfa concentrate is added into the glass containers that are then partially closed with the rubber closures. Following lyophilization a nitrogen blanket overlaying the lyophilized cake displaces oxygen, the closures are fully seated and the container closure assembly conserved with aluminum seals with a plastic flip-top. A flow diagram of the manufacturing process for production of ATryn® is presented in FIG. 17 showing in-process and final product controls for a fill/finish operation.

Receipt of Bulk Drug Substance

Individual lots of bulk antithrombin alfa are shipped from the purification site to the fill-finish site in a stainless steel vessel. Upon receipt, the condition and integrity of the vessel is inspected, the incoming documentation reviewed and the vessel subsequently stored at 2 to 8° C. until needed for the filling operation. An in-process sample is taken as outlined in Table 20. Recombinant Antithrombin alfa can be stored at 2 to 8° C. for up to 26 weeks prior to filling.

TABLE 20
In-process Testing of Antithrombin alfa

| Test | Method | Specifications |
|---|---|---|
| Identification | ELISA | Antithrombin |
| pH | Potentiometric | 6.8 to 7.2 |
| Specific Gravity | Pycnometer | Report Result |
| Bioburden | Membrane filtration | ≦2 CFU/10 mL |

Preparation of Components for Filling
Containers

Type I glass containers are rinsed with WFI and placed in clean stainless steel cassettes covered with a lid and depyrogenated. The depyrogenated containers are inventoried in clean storage until needed for the filling operation.

Rubber Closures

Ready to sterilize (RTS) type I rubber closures are autoclaved and the sterilized closures inventoried in clean storage until needed for the filling operation.

Filling

One or more lots of antithrombin alfa may be used for the production of a lot of ATryn®. Antithrombin alfa is aseptically filtered into a sterile transfer vessel and a sample of the solution collected for in-process testing prior to filtration into the surge filling tank. The surge vessel is moved adjacent to the filling machine. Filters are pre and post-use integrity tested.

An aseptic connection is made between the surge tank and the filling machine. Solution is expressed until all air has escaped from the filling heads. The target fill weight is determined based on the specific gravity of the solution multiplied by the target fill volume of 10.5 mL. Prior to starting the filling operation, two (2) sets of four (4) containers will pass a fill volume check. The filling machine then fills containers to a target of 10.5 mL of solution through a quadruple filler head. Fill weight checks are performed on a minimum of four containers per every 300 to 400 containers including containers from the beginning, middle and end of filling. The filled containers are partially closed with rubber closures and positioned into a tray-loader. Once the tray is filled, a four-sided stainless steel ring is inserted around the containers.

Lyophilization

Trays of filled and partially closed containers are transferred to the lyophilizer and loaded onto the shelves using the four sided rings. Upon completion of the vial loading, the lyophilizer door is closed and the programmed lyophilization cycle is started. Lyophilization cycle control is achieved using temperature probes and sensors which monitor product temperature, shelve temperature and vacuum level. Refer to Table 27 for the lyophilization cycle recipe. At completion of the lyophilization cycle the containers are fully closed and the lyophilizer door opened.

Container Closure Sealing

The fully closed containers are transferred to the capper where aluminum bridge seals are crimped onto the containers with plastic flip-off caps. After the capping, the containers are coded with the lot number. The 20 mm torque parameters are controlled between 0.300 and 0.600 Newton-Meters. (Note: Recorded Torque Values X 10⁻³=Value in Newton-Meters).

Heat Treatment

Sealed containers of ATryn® are loaded into a convection oven that is programmed to heat the product containers to a preset target temperature of 80° C. This temperature is maintained for 74 hours. Containers are removed from the oven upon completion of the cycle and stored in quarantine at 2 to 8° C. until finished product testing is complete and the lot is certified to meet specifications and released for packaging and labeling. Refer to Table 21 for the Heat Treatment Cycle.

TABLE 21
Heat Treatment Oven Cycle

| Step | Action | Rate |
|---|---|---|
| 1 | Ramp Temperature from ambient to 70° C. | 25° C. per hour |
| 2 | Hold at 70° C. for 3 hours | N/A |
| 3 | Ramp Temperature form 70° C. to 80.3° C. | 3° C. per hour |
| 4 | Hold at 80.3° C. for 74 hours | N/A |
| 5 | Ramp Temperature from 80.3° C. to 50° C. | 5° C. per hour |
| 6 | Ramp Temperature from 50° C. to 38° C. | 2° C. per hour |

Controls of Critical Steps and Intermediates

The target fill volume is set at 10.5 mL (gravitometrically) with an acceptable range of 10.11 to 10.80 mL. The specific gravity determined in the pre-filtration sample is used to convert mass to volume. Manual fill checks are performed approximately once every three to four hundred containers. Following lyophilization, samples are collected for analyses as shown in Table 22.

TABLE 22
Test Methods and Specifications for Lyophilized Product

| | | |
|---|---|---|
| Assay | Method | Specifications |

| | | |
|---|---|---|
| Activity | Enzymatic Hydrolysis | 110 to 260 IU/mL |
| Aggregation | HP-SEC | ≦5% |
| Appearance | Visual | White to off-white cake or |
| | | powder |
| Concentration | RP-HPLC | 22 to 29 mg/mL |
| Moisture | Karl Fisher | 0.5 to 2.5% |
| Purity | RP-HPLC | ≧98% |
| Purity | SDS PAGE | ≧95% |

Process Validation and/or Evaluation

Process validation for ATryn® manufacturing was performed in accordance with current European and U.S. regulations and guidelines. For equipment qualification, a traditional Installation Qualification (IQ), Operational Qualification (OQ) and Performance Qualification (PQ) approach has been taken. All equipment received an IQ, OQ and where applicable, PQ using worst case testing scenarios for this process.

TABLE 23
Summary of Performance Qualification Studies

| System/ | |
|---|---|
| Equipment/Process | PQ Evaluation(s) |
| Sterilizing Filters | Bacterial retention, extractables |
| Filling Process | Media fill, fill weight consistency, cleaning |
| Lyophilizer | Lyophilization process, cleaning |
| Steam Sterilizer | Filter/Fill set up and rubber closure sterilization |
| | using Biological Indicators and F₀ calculations |
| Dry Heat Oven | Endotoxin challenge using containers |
| Heat Inactivation Oven | Product quality, container closure integrity |

The foregoing is not intended to have identified all of the aspects or embodiments of the invention nor in any way to limit the invention. The accompanying drawings, which are incorporated and constitute part of the specification, illustrate embodiments of the invention, and together with the description, serve to explain the principles of the invention.

All publications and patent applications mentioned in this specification are herein incorporated by reference to the same extent as if each independent publication or patent application is specifically indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

### LITERATURE CITED AND INCORPORATED BY REFERENCE

1. Aubry, M. et al., (1972) Kinetics of the Inactivation of Human and Bovine Trypsins and Chymotrypsins by Alpha1-Proteinase Inhibitor and of their Reactivation by Alpha-2-Macroglobulin, CLIN CHIM ACTA; 78: 371-380.
2. Baguisi A, (1999) et al., Production of Goats by Somatic Cell Nuclear Transfer, NATURE BIOTECH; 17: 456-461.
3. Baker W F Jr., (1989) Clinical Aspects Of Disseminated Intravascular Coagulation: A Clinician's Point Of View, SEMIN THROMB HEMOST. 15(1):1-57.
4. Barrowcliffe, T. W. et al., (1983) Studies Of The Heterogeneity Of Antithrombin III Concentrates, BRITISH J. HAEMOTOLOGY 55:37-46.
5. Bauer, K. and Rosenberg, R. D. (1991) Role of Antithrombin III as a Regulator of In Vivo Coagulation. SEMINARS IN HEMATOLOGY 28(1):10-18.
6. Biescas, H., et al., (1998) Characterization and viral safety validation study of a pasteurized therapeutic concentrate of antithrombin III obtained through affinity chromatography. HAEMATOLOGICA 83:305-311.
7. Bjork, I., et al., (1992) Decreased affinity of recombinant antithrombin for heparin due to increased glycosylation. BIOCHEM. J. 286:793-800.
8. Boldrin M., et al., Kinetics Of Antithrombin III Activity In The Plasma After Administration Of Recombinant Human Antithrombin III In The Cynomolgus Monkey. (internal report to GTCB).
9. Broker M. et al., (1987) Expression Of Human Antithrombin III In Saccharomyces Cerevisiae And Schizosaccharomyces Pombe. BIOCHIMICA ET BIOPHYSICA ACTA. 908(3):203-13.
10. Chang, W-S. W. and Harper, P. L. (1997) Commercial Antithrombin Concentrate Contains Inactive L-Forms of Antithrombin. THROMBOSIS & HAEMOSTASIS 77:323-328.
11. Cooper, P C, Cooper, S., Kitchen, S., and Makris, M. (2003) Characterisation Of A New Therapeutic Recombinant Human Antithrombin Concentrate. (INTERNATIONAL SOCIETY OF THROMBOSIS & HEMOSTASIS, Birmingham, UK July 2003).
12. Cowan, P., et al., (2002) Protective Effects of Recombinant Human Antithrombin III In Pig-To-Primate Renal Xenotransplantation. AM J TRANSPLANT 2(6):520-525.
13. Dalsgaard Nielsen, J. (1998) The Effect of Antithrombin on The Systemic Inflammatory Response In Disseminated Intravascular Coagulation. BLOOD COAGULATION AND FIBRINOLYSIS 9(suppl 3):S11-S15.
14. Dickneite, G. and Paques, E-P (1993) Reduction Of Mortality With Antithrombin III In Septicemic Rats: A Study Of Klebsiella Pneumoniae Induced Sepsis. THROMBOSIS AND HAEMOSTASIS 69:988-102.
15. Dunzendorfer, S., et al., (2001) Cell-Surface Heparin Sulfate Proteoglycan-Mediated Regulation Of Human Neutrophil Migration By The Serpin Antithrombin III. BLOOD 97:1079-1085.
16. Edmunds, T., et al., (1998) Transgenically Produced Human Antithrombin. Structural And Functional Comparison To Human Plasma-Derived Antithrombin. BLOOD 91:4561-4571.
17. Fan, B., et al., (1993) Heterogeneity Of Recombinant Human Antithrombin III Expressed In Baby Hamster Kidney Cells. J. BIOL. CHEM. 268:17588-17596.
18. Franzen, L-E. and Svensson, S. (1980) Structural studies on the carbohydrate portion of human antithrombin III. J. BIOL. CHEM. 255:5090-5093.
19. Gabler et al., (1987), Principles of Tangential Flow Filtration. Applications to Biological Processing, in FILTRATION IN THE PHARMACEUTICAL INDUSTRY, pp. 453-490.
20. Groner, A., et al., (1999) Elimination of parvovirus B19 by the manufacturing process of Kybernin P. ANN. HEMATOL. 78 (Suppl. 1):A90.
21. Harper, P. L., Evans, D., and Carrell, R. W. (1997) THE POTENTIAL HAZARDS OF ANTITHROMBIN PREPARATIONS.
22. Hellstern, P., et al., (1995) In vitro characterization of antithrombin III concentrates—A single-blind study. HAEMOSTASIS 25:193-201.
23. Herczeg L. et al., Morphological Damage to the Central Nervous System (CNS) Following Open Heart Surgery, FORENSIC SCIENCE INTERNATIONAL, 79(2):103-11, (1996).
24. Hoffmann, J. N., et al., (2000). Antithrombin reduces leukocyte adhesion during chronic endotoxemia by modulation of the cyclooxygenase pathway. AMERICAN JOURNAL OF PHYSIOLOGY—CELL PHYSIOLOGY. 279:C98-C107.
25. Hoffmann, J. N., et al., (2002). Antithrombin effects on endotoxin-induced microcirculatory disorders are mediated mainly by its interaction with microvascular endothelium. CRITICAL CARE MEDICINE. 30:218-225.
26. Inoue K. et al., Incidence and Risk Factors of Perioperative Cerebral Complications: Heart Transplantation Compared to Coronary Artery Bypass Grafting and Valve Surgery, JOURNAL OF CARDIOVASCULAR SURGERY, 39(2):201-8, (1998).
27. Johnsson P. et al., Cerebral Complications After Cardiac Surgery Assessed by S-100 and NSE Levels in Blood, JOURNAL OF CARDIOTHORACIC & VASCULAR ANESTHESIA, 9(6):694-9, (1995).
28. Kaneider, N., Forster, E., Dunzendorfer, S., Djanani, A., Egger, P., and Wiedermann, C. (2002) Syndecan-4-dependent inhibition of endotoxin-induced endothelial adherence of neutrophils by antithrombin. European Society of Intensive Care Medicine Presentation, Barcelona, Spain.
29. Kessler, C. M, Tang, Z., Jacobs, H. M., and Szymanski, L. M. (1997) The suprapharmacologic dosing of antithrombin concentrate for staphulococcus aureus-induced disseminated intravascular coagulation in guinea pigs: Substantial reduction in mortality and morbidity. BLOOD 89:4393-4401.
30. Konkle, B., Bauer, K., Weinstein, R., Greist, A., Holmes, H., and Bonfiglio, J. (2003) Use of recombinant human antithrombin (AT) in patients with congenital AT deficiency undergoing surgical procedures. Accepted for publication in TRANSFUSION.
31. Kounnas M Z, Church F C, Argraves W S, and Strickland D K. (1996) Cellular internalization and degradation of antithrombin III-thrombin, heparin cofactor II-thrombin, and alpha 1-antitrypsin-trypsin complexes is mediated by the low density lipoprotein receptor-related protein. J BIOL CHEM. 271:6523-9.
32. Lahiri, B., Bagdasarian A., Mitchell B., Talamo R C and Colman R W. (1976) Antithrombin-Heparin Cofactor: An Inhibitor of Plasma Kallikrein. ARCHIVES BIOCHEM BIOPHYS. 175: 737-747.
33. Lebing, W., Hammond, D., Wydick, J., and Baumbach, G. (1994) A highly purified antithrombin III concentrate prepared from human plasma fraction IV-1 by affinity chromatography. VOX SANG 67(2): 117-24.
34. Levy, J. H., Despotis, G. J., Szlam, F., Olson, P., and Meeker, D. (2002) Recombinant human transgenic antithrombin in cardiac surgery: A dose-finding study. ANESTHESIOLOGY 96:1095-1102.
35. Levy, J., Weisinger, A., Ziomek, C., and Echelard, Y. (2001) Recombinant antithrombin: production and role in cardiovascular disorder. SEMINARS IN THROMBOSIS & HAEMOSTASIS 27(4):405-416.
36. Levy, J. H., Montes, F., Szlam, F., and Hillyer, C. D. (2000) The in vitro effect of antithrombin III on the activated coagulation time in patients on heparin therapy. ANESTH ANALG. 90:1076-1079.
37. Lu, W., Mant, T G K, Levy, J. H. and Bailey, J. M. (2000) Pharmacokinetics of recombinant transgenic antithrombin in volunteers. ANESTH ANALG 90:531-534.
38. Marzo A, Ceppi-Monti N, Giusti A, Abbiati G, and Parenti M. (2002) Pharmacokinetic Behaviour of Antithrombin III Following Intravenous Infusion In Healthy Volunteers. ARZNEIMITTELFORSCHUNg. 52(3):187-93.
39. Meagher, J. L., Beechem, J. M., Olson, S. T., and Gettins, P. G. W. (1998) Deconvolution of The Fluorescence Emission Spectrum of Human Antithrombin and Identification of the Tryptophan Residues that are Responsive to Heparin Binding. J. BIOL. CHEM. 273:23283-23289.
40. Menache, D., (1991) Antithrombin III: Introduction. SEMINARS IN HEMATOLOGY 28(1):1-2.
41. Menache, D., B. J. Grossman, and C. M. Jackson, (1992) Antithrombin III: physiology, deficiency, and replacement therapy. TRANSFUSION 32(6):580-588.
42. Meng L, et al., (1997) Rhesus Monkeys Produced by Nuclear Transfer, BIOL REPROD. August; 57(2):454-9.
43. Minnema M C, Chang A C, Jansen P M, Lubbers Y T, Pratt B M, Whittaker B G, Taylor F B, Hack C E, and Friedman B. (2000) Recombinant human antithrombin III improves survival and attenuates inflammatory responses in baboons lethally challenged with Escherichia coli. Blood. 95(4):1117-23.
44. Mizouchi, T., Fuji, J., Kurachi, K, and Kobata, A., (1980) Stuctural studies of the carbohydrate moiety of human antithrombin III. ARCHIVES OF BIOCHEM & BIOPHYS. 203:458-465.
45. Mortensen, R, et al., (1992) Production of Homozygous Mutant ES Cells with a Single Targeting Construct, MOL. CELL. BIOL. 12, 2391-2395.
46. Munzert, E., Muthing, J., Buntemeyer, H., and Lehmann, J. (1996) Sialidase Activity In Culture Fluid Of Chinese Hamster Ovary Cells During Batch Culture And Its Effect On Recombinant Human Antithrombin III Integrity. BIOTECHNOL. PROG. 12:559-563.
47. Murakami, K., Cox, R., Hawkins, H., Schmalstieg, F., Traber, L., Prough, D., Herndon, D., Traber, L., and Traber, D. (2002) Recombinant Antithrombin (Rhat) Inhibits Airway Obstruction After Smoke Inhalation In Sheep. FASEB JOURNAL. 16(4):A76).
48. Murakami, K., Katahira, J., Traber, L., and Traber, D. (2001) Effect Of Anticoagulants on Septic Changes After Smoke Inhalation In Sheep. FASEB JOURNAL. 15(5):A854.
49. Murakami, K., McGuire, R., Cox, R., Jodoin, J., Schmalstieg, F., Traber, L., Hawkins, H., Herndon, D., and Traber, D. (2003) Recombinant Antithrombin Attenuates Pulmonary Inflammation Following Smoke Inhalation And Pneumonia In Sheep. CRIT. CARE MED; 31(2):577-583.
50. Murano, G., L. Williams, and M. Miller-Andersson, (1980) Some Properties of Antithrombin III and its Concentration in Human Plasma. THROMB RES. 18(1-2):259-262.
51. Murkin J M., Etiology snd Incidence of Brain Dysfunction After Cardiac Surgery, JOURNAL OF CARDIOTHORACIC & VASCULAR ANESTHESIA. 13(4 Suppl 1): 12-7; discussion 36-37, (1999).
52. Neviere, R., Tournoys, A., Mordon, S., Marechai, X., Song, F., Jourdain, M., and Fourrier, F. (2001) Antithrombin Reduces Mesenteric Venular Leukocyte Interactions And Small Intestine Injury In Endotoxemic Rats. SHOCK 15(3):220-225.
53. Nordenman, B., et al, (1977) The size and shape of human and bovine antithrombin III. EUR. J. BIOCHEM. 78:195.
54. Olson, S. T., et al., (1997) Effect of Individual Carbohydrate Chains of Recombinant Antithrombin on Heparin Affinity and on the Generation of Glycoforms Differing in Heparin Affinity, ARCHIVES OF BIOCHEM & BIOPHYS. 341:212-221.
55. Ongeri E M, et al., (2001) Development of Goat Embryos After In Vitro Fertilization and Parthenogenetic Activation by Different Methods, THERIOGENOLOGY June 1; 55(9):1933-45.
56. Ostrovsky, L., et al., (1997). Antithrombin III prevents and rapidly reverses leukocyte recruitment in ischemia/reperfusion. CIRCULATION 96:2302-2310.
57. Petersen, T., et al., (1979) Primary Structure of Antithrombin III, in The Physiological Inhibitions of Blood Coagulation and Fibrinolysis, D. W. Collen B and Verstraete M, Editor. Elsevian/North Holland Biochemical Press; Amsterdam: pp. 43-54.
58. Pratt C W and Church F C. (1991) Antithrombin: Structure And Function. SEMINARS IN HEMATOLOGY 28 (1):3-9.
59. Porter, ed., HANDBOOK OF INDUSTRIAL MEMBRANE TECHNOLOGY, (Noyes Publications, Park Ridge, N.J., 1998) pp. 160-176.
60. REMINGTON'S PHARMACEUTICAL SCIENCES (16^{th} ed., Osol, A., editor; Mack, Easton Press. (1980)).
61. Richards, S., et al., (2003) Assessment Of Immunogenicity Of Recombinant ATIII In Human Clinical Studies. ANNALS OF HEMATOLOGY 82(Suppl 1):S72.
62. Risberg, B. (1998) Antithrombin. Facts And New Hypotheses. BLOOD COAGULATION AND FIBRINOLYSIS 9 (Suppl 3):S3-S6.
63. Roemisch J, Gray E, Hoffmann J N, and Wiedermann C J. (2002) Antithrombin: a new look at the actions of a serine protease inhibitor. BLOOD COAGUL FIBRINOLYSIS. 13(8):657-70.
64. Rosenberg, Robert D. (1989). Biochemistry Of Heparin Antithrombin Interactions, And The Physiologic Role Of This Natural Anticoagulant Mechanism. AM. J. MED. 87 (Suppl 3B):2S-9S.
65. Sambrook et al., MOLECULAR CLONING—A LABORATORY MANUAL, Cold Spring Harbor Laboratory Press, (2nd Edition) (1989).
66. Schinzel, H. and Weilemann, L. S. (1998) Antithrombin substitution therapy. BLOOD COAGULATION AND FIBRINOLYSIS 9 (Suppl 3):S17-S22.
67. Schreuder, H. A., de-Boer, B., Dijkema, R., Mulders, J., Theunissen, H. J., Grootenhuis, P. D. and Hol, W. G. (1994) The intact and cleaved human antithrombin III complex as a model for serpin-proteinase interactions. NAT. STRUCT. BIOL. 1:48.
68. Soloway, H. B., Christiansen, T. W., Heparin Anticoagulation During Cardiac Surgery in an AT III Deficient Patient, AM. J CLIN. PATHOL. 73: 723-5, (1980).
69. Souter, P., Gray, E. Effects of transgenic antithrombin (Genzyme) on tissue factor and cytokine productions by mononuclear cells and whole blood. (internal report to GTCB).
70. Souter P J, Thomas S, Hubbard A R, Poole S, Romisch J, and Gray E. (2001) Antithrombin inhibits lipopolysaccharide-induced tissue factor and interleukin-6 production by mononuclear cells, human umbilical vein endothelial cells, and whole blood. CRIT CARE MED. 29(1):134-9.
71. Swedenborg, J. (1998) The mechanism of action of α-and β-isoforms of antithrombin. BLOOD COAGULATION AND FIBRINOLYSIS 9(Suppl 3):S7-S10.
72. Taylor, F. B., Emerson, T. E, Jordan, R., Chang, A. K. and Blick, K. E. (1988) Antithrombin-III Prevents The Lethal Effects of Escherichia Coli Infusion in Baboons. CIRCULATORY SHOCK 26:227-235.
73. Travis, J. and G. S. Salvesen, (1983) Human Plasma Proteinase Inhibitors. ANNUAL REVIEW OF BIOCHEMISTRY 52:655-709.
74. Turk, B., Brieditis, I., Bock, S. C., Olson, S. T. and Bjork, I. (1997) The oligosaccharide side chain of Asn-135 of α-antithrombin, absent in β-antithrombin, decreases the heparin affinity of the inhibitor by affecting the heparin-induced conformational change. BIOCHEM. 36:6682-6691.
75. Van Aken, H., et al., (2000) Recombinant human ATIII (rhATIII) Improves Anticoagulation in Heparin Resistant Patients Undergoing Cardiopulmonary Bypass. (European Association of Cardiothoracic Anesthesiologists Annual Meeting, Aarhus, Denmark June 2000).
76. Van Boven, H. H. and Lane, D. A. (1997) Antithrombin and its Inherited Deficiency States. SEMINARS IN HEMATOLOGY 34:188-204.
77. Van Patten, S., et al., (1999), Oxidation of methionine residues in antithrombin. Effects on biological activity and heparin binding, J. BIOL. CHEM. 274:10268-10276.
78. Videm V. et al., Heparin-Coated Cardiopulmonary Bypass Equipment. I. Biocompatibility Markers And Development Of Complications In A High-Risk Population, JOURNAL OF THORACIC & CARDIOVASCULAR SURGERY. 1117(4):794-802, (1999).
79. Wilmut I, et al., (2002) Somatic Cell Nuclear Transfer, NATURE 10; 419(6907):583-6.
80. Wilmut I, et al., (1997) Viable Offspring Derived From Fetal and Adult Mammalian Cells, NATURE February 27; 385(6619):810-3.
81. Zettlmeissl, G., et al., (1989) Characterization Of Recombinant Human Antithrombin III Synthesized In Chinese Hamster Ovary Cells. J. BIOL. CHEM. 264:21153-21159.
82. Ziomek, C., et al., (2003) Viral And Prion Safety Of Recombinant Human Antithrombin. ANNALS HEMATOLOGY 82 (Suppl 1):S95.
83. Zou X, et al., (2002) Generation of Cloned Goats (Capra Hircus) from Transfected Foetal Fibroblast Cells, The Effect of Donor Cell Cycle, MOL REPROD DEV.; 61: 164-172.

### PATENTS CITED AND INCORPORATED BY REFERENCE

1. Meade et al., U.S. Pat. No. 5,750,172.
2. Meade et al., U.S. Pat. No. 4,873,316.
3. Stice et al., U.S. Pat. No. 5,945,577.
4. Meade, et al., U.S. Pat. No. 5,827,690.
5. van Reis et al., U.S. Pat. No. 5,256,294; Tangential Flow Filtration Process And Apparatus.
6. Lenk et al., U.S. Pat. No. 5,948,441; Method for Size Separation of particles.
7. van Reis et al., U.S. Pat. No. 5,490,937; Tangential Flow Filtration Process and Apparatus.
8. Marinaccio et al., U.S. Pat. No. 4,888,115; Cross-Flow Filtration.
9. Couto et al., U.S. patent application Ser. No. 10/635,117

What is claimed is:

1. A method for purifying a molecular species of interest from a feedstream, comprising:
(a) Filtering said feedstream by a tangential-flow filtration process (TFF) to produce a TFF permeate;
(b) Cycling said TFF permeate through a closed loop system until at least 50% of the said molecular species of interest is captured wherein said closed loop system further comprises a Heparin-affinity column;
(c) Nanofiltering said TFF/heparin eluate viral removal such that potential viral adventitious agents are removed;
(d) Removing unwanted molecular contaminants through the use of an anion exchange column;
(e) Utilizing a hydrophobic interaction column to eliminate unwanted or variant forms of said molecular species of interest; and,
(f) formulating said molecular species;
(g) lyophilizing said molecular species; and,
(h) Heating the purified lyophilized molecular species of interest to inactive viruses.

2. The method of claim 1, further comprising lyophilizing said molecular species of interest after eluting this product from said hydrophobic interaction column.

3. The method of claim 1, wherein said heating further comprises a virus inactivation step providing dry heat of 80° C. for at least 72 hours.

4. The method of claim 1, wherein said heparin affinity column is a Heparin-Hyper D column.

5. The method of claim 1, wherein said TFF permeate is cycled through a closed loop system until at least 60% of said molecular species of interest is captured from a feedstream.

6. The method of claim 1, wherein said TFF permeate is cycled through a closed loop system until at least 90% of said molecular species of interest is captured from a feedstream.

7. The method of claim 1, wherein said TFF permeate is cycled through a closed loop system for at least 5 volume cycles.

8. The method of claim 1, wherein said TFF permeate is cycled through a closed loop system for at least 8 volume cycles.

9. The method of claim 1, wherein after said TFF permeate is cycled through said heparin affinity column the retentate molecular species of interest is washed and then eluted with a first buffer.

10. The method of claim 9, wherein said first buffer is a salt buffer further comprising a sodium chloride buffer.

11. The method of claim 10, wherein said salt buffer is 2.5 M sodium chloride

12. The method of claim 1, wherein said anion column is a sepharose column.

13. The method of claim 12, wherein said sepharose column is a ANX-Sepharose column.

14. The method of claim 13, wherein after the use of said ANX-Sepharose column said molecular species of interest is eluted from said ANX-Sepharose column with a second buffer.

15. The method of claim 14, wherein said second buffer is 0.32 M sodium chloride.

16. The method of claim 1, wherein after said anion exchange column a product eluate is collected and conditioned with sodium citrate.

17. The method of claim 16, wherein said conditioned product eluate is applied to a Methyl HyperD column and eluted with a third buffer.

18. The method of claim 17, wherein said third buffer is a sodium citrate buffer.

19. The method of claim 1, wherein the final formulation of the said conditioned product eluate is achieved by concentration and diafiltration into a citrate, glycine, sodium chloride buffer.

20. The method of claim 19, wherein the final protein concentration of the composition ranges from 20 IU/ml to 200 IU/ml.

21. The method of claim 1, wherein said heat treatment further comprises heat treating lyophilized molecular species of interest at 80° C. for 72 hours in a viral inactivation step.

22. The method of claim 1, wherein during said tangential flow filtration process the flux is maintained at a level ranging from about 5 to 100% of transition point flux in the pressure-dependent region of the flux versus TMP curve.

23. The method of claim 22, wherein transmembrane pressure is held substantially constant along the membrane at a level no greater than the transmembrane pressure at the transition point of the filtration, whereby said molecular species of interest is selectively separated from said feedstream such that said molecular species of interest retains its biological activity.

24. The method of claim 1, wherein said tangential-flow filtration process through a filtration membrane having a pore size that separates said molecular species of interest from said feedstream.

25. The method of claim 24, wherein said filtration membrane has a pore size of between 200 and 700 kD.

26. The method of claim 25, wherein said filtration membrane has a pore size of 500 kD.

27. The method of claim 26, wherein said filtration membrane is a 500 kD hollow fiber membrane.

28. The method of claim 1, wherein prior to entering the purification process the feedstream is diluted with an equal volume of an EDTA buffer.

29. The method of claim 1, wherein said molecular species of interest is an antithrombin protein.

30. The method of claim 1, wherein said purity of said molecular species of interest was at least 90%.

31. The method of claim 1, wherein said purity of said molecular species of interest was greater than 99%.

32. The method of claim 1, wherein said physiological activity of said molecular species of interest was at least 90%.

33. The method of claim 1, wherein said physiological activity of said molecular species of interest was greater than 99%.

34. The method of claim 1, wherein the purity of said molecular species is determined by SDS-PAGE.

35. The method of claim 1, wherein the purity of said molecular species is determined by reverse phase-HPLC.

36. The method of claim 29, wherein said molecular species of interest is a recombinant antithrombin protein.

37. The method of claim 36, wherein said molecular species of interest is a transgenically produced recombinant antithrombin protein.

38. The method of claim 1, wherein all filtration stages are ultrafiltrations.

39. The method of claim 1, wherein said feedstream is milk

40. The method of claim 1, wherein said feedstream is a cell lysate solution.

41. The method of claim 1, wherein said molecular species of interest is a biopharmaceutical.

42. The method of claim 39, wherein the condition of said milk is selected from one of the following states:
a) raw;
b) diluted;
c) treated with a buffer solution;
d) chemically treated; and
e) partially evaporated.

43. The method of claim 1, wherein said fractionation step utilizes ceramic filtration membranes.

44. The method of claim 1, wherein said clarification step utilizes ceramic filtration membranes.

45. The method of claim 1, wherein said fractionation step utilizes polymeric filtration membranes.

46. The method of claim 1, wherein said clarification step utilizes polymeric filtration membranes.

47. The method of claim 1, wherein said fractionation step utilizes cellulose filtration membranes.

48. The method of claim 1, wherein said clarification step utilizes cellulose filtration membranes.

49. The method of claim 1, further comprising optimizing systematic parameters.

50. The method of claim 49, wherein said systematic parameters include temperature, feedstream flow velocity, transmembrane pressure, feedstream concentration and diafiltration volume.

51. The method of claim 50, further comprising optimizing systematic parameters for the production of recombinant human antithrombin.

52. The method of claim 1, wherein said molecular species of interest are biological entities selected from the group consisting of proteins, polypeptides, peptides and glycoproteins,

53. The method of claim 1, wherein the optimal temperature range is from 15° C. to 50° C.

54. The method of claim 1, wherein the optimal temperature range is from 20° C. to 35° C.

55. The method of claim 1, wherein the optimal temperature range is from 25° C. to 29° C.

56. The method of claim 1, wherein the feedstream flow velocity is from 10 cm/sec to 100 cm/sec.

57. The method of claim 1, wherein the feedstream flow velocity is from 20 cm/sec to 60 cm/sec.

58. The method of claim 1, wherein the feedstream flow velocity is from 25 cm/sec to 45 cm/sec.

59. The method of claim 1, wherein the transmembrane pressure ranges from 2 psi to 40 psi.

60. The method of claim 1, wherein the transmembrane pressure ranges from 5 psi to 30 psi.

61. The method of claim 1, wherein the transmembrane pressure ranges from 10 psi to 20 psi.

62. The method of claim 39, wherein the feedstream concentration is from 0.25× to 4× natural milk.

63. The method of claim 39, wherein the feedstream concentration is from 0.5× to 3× natural milk.

64. The method of claim 39, wherein the feedstream concentration is from 1.0× to 2× natural milk.

65. The method of claim 1, wherein the diafiltration volume range is from 1× to 20× the volume of concentrated retentate.

66. The method of claim 1, wherein the diafiltration volume range is from 3× to 15× the volume of concentrated retentate.

67. The method of claim 1, wherein the diafiltration volume range is from 5× to 10× the volume of concentrated retentate.

68. The method of claim 39, wherein said milk is treated with a solution selected from the group consisting of:
a) water;
b) a buffered aqueous salt solution;
c) chelating agent;
d) acid solution; and
e) alkali solution.

69. The method of claim 1, further comprising filtering the filtrate from the filtration in a second tangential-flow filtration stage through a membrane having a smaller pore size than the membrane used in the first filtration stage, and recycling the filtrate of this second filtration stages back to the first filtration stage, whereby the process is repeated.

70. A pharmaceutical composition according to the method of claim 1, wherein the reconstitution media is selected from any one of polysorbate 20, polysorbate 21, polysorbate 40, polysorbate 60, polysorbate 61, polysorbate 65, polysorbate 80, polysorbate 81, polysorbate 85, polysorbate 120; and, human albumin.

71. A pharmaceutical composition according to claim 70, wherein the reconstitution media is polysorbate 80.

72. A pharmaceutical composition according to claim 70, wherein the reconstitution media is human albumin.

73. A method of purifying a recombinant antithrombin III (rhAT) or a fragment thereof from a feedstream, comprising:
Solubilizing said rhAT from a feedstream utilizing a tangential flow filtration process;
Washing said filtrate on a membrane with a PBS solution wherein said rhAT or fragment thereof remains in the retentate on the purification column;
Adding an aqueous solution to said rhAT remaining in the retentate to solubilize it;
Eluting said rhAT from said purification column; and,
Purifying out said rhAT from elution.

74. A method for purifying a molecular species of interest from a feedstream, comprising:
a) Filtering said feedstream by a tangential-flow filtration process (TFF) to produce a TFF permeate;
b) Cycling said TFF permeate to through a closed loop system until at least 50% of the said molecular species of interest is captured wherein said closed loop system further comprises a Heparin-affinity column;
c) Collecting a first eluate from said Heparin-affinity column and processing said first eluate through a first concentration step and a first diafiltration step;
d) Transferring said first eluate into a downstream processing and formulation area thereafter again transferring said first eluate after said first concentration step and said first diafiltration step to a ion exchange chromatography column to generate a second eluate;
e) Removing unwanted molecular contaminants through the use of an anion exchange column;
f) Processing said second eluate through an anionic exchange chromatography column;
g) Nanofiltering said TFF permeate ion step for viral removal such that potential adventitious agents are removed;
h) Processing said second eluate through a second concentration step and a second diafiltration step to generate a third eluate;
i) Transferring said product eluate through a nanofilter capable of removing viruses from said third eluate;
j) Utilizing a hydrophobic interaction column to eliminate unwanted or variant forms of said molecular species of interest;
k) Processing said product eluate through a third concentration step and a third diafiltration step to generate a product eluate;
l) Loading said product eluate on a anion column and thereafter eluting with a buffer; and,
m) Heating the purified molecular species of interest to inactive viruses.

75. The process of claim 74, wherein said molecular species of interest is rhAT.

76. The molecular species of interest produced by the process of claim 74.

77. The molecular species of interest produced by the process of claim 1.

78. The molecular species of claim 77, wherein said molecular species of interest is used therapeutically.

79. The therapeutic conditions of claim 78, wherein the therapeutic condition treated is selected from the group consisting of: a hereditary rhAT deficiency; DIC, burns, heparin resistance, neurocognitive deficit due to CABG surgery; and, sepsis.

80. The process of claim 1, wherein the resultant purified molecular species of interest is more than 90% from prion contamination found in normal milk.

81. The process of claim 1, wherein the resultant purified molecular species of interest is more than 90% from prion contamination found in transgenic milk.

82. The process of claim 1, wherein the resultant purified molecular species of interest is more than 90% from viral contamination found in normal milk.

83. The process of claim 1, wherein the resultant purified molecular species of interest is more than 90% from viral contamination found in transgenic milk.
